(19) United States
(12) Patent Application Publication
(10) Pub. No.: US 2020/0216824 A1
(43) Pub. Date: Jul. 9, 2020

(54) REGULATING PROTEIN SPONTANEOUS RUPTURES

(71) Applicant: Bowling Green State University, Bowling Green, OH (US)

(72) Inventor: H. Peter Lu, Bowling Green, OH (US)

(73) Assignee: Bowling Green State University, Bowling Green, OH (US)

(21) Appl. No.: 16/731,648

(22) Filed: Dec. 31, 2019

Related U.S. Application Data
(60) Provisional application No. 62/787,886, filed on Jan. 3, 2019.

(51) Int. Cl. C12N 9/02; C12N 9/12; C07K 14/47

(52) U.S. Cl. CPC C12N 9/0075; C12Y 114/13039; C07K 14/4711; C12Y 207/06003; C07K 14/4728; C12N 9/1235

(57) ABSTRACT

Protein rupture under compressive forces can be regulated by cations. More specifically, pico-Newton forces can cause rupture of protein molecules, as shown in examples with calmodulin (CaM) and tau proteins, among others. However, rupture does not occur in the presence of various concentrations of cation(s), thus elucidating new targets for disease therapy and providing therapies for neurodegenerative diseases or other conditions involving protein misfolding, dysfunction, or aggregation.

### RELATED APPLICATIONS

This application claims priority to United States Provisional Application No. 62/787,886 filed under 35 U.S.C. § 111(b) on Jan. 3, 2019, the disclosure of which is incorporated herein by reference in its entirety for all purposes.

### STATEMENT REGARDING FEDERALLY SPONSORED RESEARCH

This invention was made with no government support. The government has no rights in this invention.

### BACKGROUND

Proteins perform many, if not most, functions in biology, from cellular message signaling to gene transcription. A protein's three-dimensional structure is intimately related to the function of the protein. The three-dimensional structure is often formed by so-called “weak” bonds, such as, but not limited to, hydrogen bonds and van der Waals forces. Via disruption of these weak bonds, proteins often undergo dynamic transitions including folding, unfolding, and conformational changes to perform biological functions in living cells. Beyond the static structure-function relationship, the dynamic relationship between protein conformations and their functions has been extensively studied. Mechanical force interaction and fluctuation of both the amplitude and the vector orientations on proteins can be significant in a living cell, being originated from the biological component movements and interactions, such as, molecular aggregation, molecular partition crowding, hydrodynamic stress, cell osmotic pressure, and cell entropic surface tension.

Disruption of the three-dimensional structure can impact the function of the protein dramatically. For example, proteins can refold improperly after disruption, forming a structure that functions differently or not at all. Clusters of proteins can be disrupted and then refold back to an entangled structure state with unexpected properties. Such entanglements can be found with tau proteins, which are thought to play a role in neurodegenerative diseases such as Alzheimer's or dementia, aggregating into a fibril. In addition, disruption in chromatin or histone proteins can trigger uncontrolled or random changes to gene expression, which can lead to gene translational diseases such as cancer.

Manipulating protein conformations for exploring protein structure-function relationships has shown a great promise. And, although protein conformational changes under pulling force manipulation have been extensively studied, protein conformation changes under a compressive force have not been explored quantitatively. The latter is even more biologically significant and relevant in revealing the protein functions in living cells, associated with protein crowdedness, distribution fluctuations, and cell osmotic stress. There is a need in the art for methods of regulating conformational changes of protein under compressive forces.

### SUMMARY

Provided herein is a method for preventing or reducing rupture of a protein from a compressive force, the method comprising exposing the protein to a concentration of a cation effective to prevent or reduce rupture of the protein from the compressive force. In certain embodiments, the protein is present in a human cell. In certain embodiments, the protein is a globular protein. In certain embodiments, the the protein is selected from the group consisting of tau, HPPK, nitric oxide synthase (NOS), and calmodulin (CaM).

In certain embodiments, the cation is selected from the group consisting of metallic cations and organic cations. In certain embodiments, the cation comprises Ca²⁺. In certain embodiments, the cation comprises Mg²⁺. In certain embodiments, the cation comprises Zn²⁺. In certain embodiments, the concentration of the cation is a micromolar concentration. In certain embodiments, the concentration of the cation is a millimolar concentration.

In certain embodiments, the compressive force is about 12 pN. In certain embodiments, the compressive force is about 35 pN. In certain embodiments, the compressive force is about 60 pN.

In certain embodiments, the protein is in an aqueous solution. In particular embodiments, the aqueous solution is present in a living cell of a subject. In particular embodiments, the subject is selected from the group consisting of human, mammal, and other animal In particular embodiments, the subject has a neurodegenerative disease or other condition involving protein misfolding, dysfunction, or aggregation.

Further provided is a method of causing a spontaneous protein rupture, the method comprising applying a picoNewton compressive force to a protein to cause the protein to spontaneously rupture. In certain embodiments, the picoNewton compressive force is applied by a tip of an atomic force microscope. In certain embodiments, the applied compressive force is at least 20 pN. In certain embodiments, the applied compressive force is at least 25 pN. In certain embodiments, the applied compressive force is at least 60 pN. In certain embodiments, the applied compressive force is at least 75 pN.

Further provided is an aqueous solution comprising a protein with a rupture threshold exposed to a compressive force in excess of the rupture threshold; and a concentration of a cation; wherein the concentration of the cation is sufficient to prevent rupture of the protein upon exposure to the compressive force in excess of the rupture threshold, and the protein is in a non-ruptured state.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the U.S. Patent and Trademark Office upon request and payment of the necessary fees.

FIGS. 1A-1D: Schematics of the AFM-FRET nanoscopy setup and single protein molecule rupture described in the examples herein.

FIG. 1E: Mechanical force curve of the AFM tip interaction with a single HPPK molecule and a typical experimental compressive force curve trajectory recording single-molecule HPPK protein ruptures under the compressive force.

FIGS. 2A-2C: Schematics of the structure of HPPK (FIG. 2A) and CaM (FIG. 2B) molecules. FIG. 2C shows an AFM image of a single HPPK molecule on a cover glass surface and the topography of the encircled single HPPK protein molecule along the yellow line.

FIG. 3: Graph illustrating the use of a AFM force spectroscopy curve to achieve protein rupture threshold force.

FIGS. 4A-4C: Force curves on different glass surfaces. FIG. 4A shows glass coated with (3-aminopropyl) trimethoxysilane. FIG. 4B shows glass coated with isobutyltrimethoxysilane. FIG. 4C shows non-modified bare glass.

FIGS. 5A-5B: Force curve of HPPL ruptures under AFM tip compressive force loading, a single-molecule force-time trajectory (FIG. 5A), and a zoomed-in view of the circled area of the force curve (FIG. 5B).

FIGS. 6A-6E: Correlated time trajectories of the AFM-FRET single-molecule protein force manipulation measurements. FIG. 6A shows the force trajectory of a pushing cycle. Time at the point of rupture is considered as time zero, before rupture (−)ve time, after rupture (+)ve time. FIG. 6B shows the force trajectory (at the zoomed-in area of FIG. 6A) correlated with FRET efficiency trajectories. FIG. 6C shows the average FRET efficiency of ten correlated trajectories with standard deviation error bar and binning time of 2 ms. FIG. 6D shows the average of FRET efficiency change with standard deviation error bar and binning time of 2 ms. FIG. 6E shows the distribution of Pearson's product-moment correlation coefficient r_{F,E} (T, n) of force and FRET efficiency near the moment of the protein rupture calculated from 13 sets of AFM-FRET correlated trajectories with 2 ms binning time.

FIGS. 7A-7B: Correlated time trajectories of force curve and a correlated single-molecule FRET trajectory correlation parameter curve for the multiple single-molecule HPPK rupture recorded time trajectories under the pictoNewton compressive force. FIG. 7A shows the force trajectory of a pushing cycle on a single CaM molecule. FIG. 7B shows the distribution of Pearson's product-moment correlation coefficient r_{F,E} (T, n) of force and FRET efficiency near the moment of the CaM protein rupture calculated from 68 sets of AFM-FRET correlated trajectories with 10 ms of binning time.

FIGS. 8A-8D: Characterization of the protein rupture threshold values measured under the compressive force loading on single HPPK molecules under different force loading rates. FIG. 8A shows the HPPK rupture threshold force distribution under 200 nm/s loading velocities. FIG. 8B shows the Apparent Loading Rate (ALR) dependent rupture threshold force distribution for HPPK. FIG. 8C shows the CaM rupture threshold force distribution under 200 nm/s loading velocities. FIG. 8D shows the apparent Loading Rate (ALR) dependent rupture threshold force distribution for CaM. The unit of ALR is pN/s.

FIGS. 9A-9D: Characterization of the single-molecule HPPK enzyme rupture threshold force value histograms under different compressive force loading rates: 200 nm/s (FIG. 9A), 400 nm/s (FIG. 9B), 1000 nm/s (FIG. 9C), and 2000 nm/s (FIG. 9D).

FIGS. 10A-10D: Characterization of HPPK rupture force and loading energy. FIG. 10A shows an example of experimental force curve. The small red circles show the point where the compressive force just exceeds 0 pN, reaches 5, 10, 15, 20, and 25 pN, respectively. FIG. 10B shows the estimated average rupture induction length after adjustment with standard deviation, assuming the atomic level contact between AFM tip and the protein occurs after the force reaches 5, 10, 15, 20, and 25 pN, respectively. FIG. 10C shows AFM piezoelectric displacement distribution, which is defined as the distance traveled by AFM piezoelectric between the start of the force loading on a targeted protein and the protein rupture. FIG. 10D shows the distribution of calculated loading energy under 200 nm/s loading velocity.

FIG. 11: Histograms of the protein HPPK rupture force and converted loading energy in stress at the protein rupture threshold force values, measured at different force loading rates.

FIGS. 12A-12B: Crystal structures of CaM, the activated form (FIG. 12A) and the non-activated form (FIG. 12B). FIG. 12A shows the crystal structure of Ca²⁺-ligated calmodulin. FIG. 12B shows the NMR structure of Apo-calmodulin in solution.

FIGS. 13A-13B: Interaction of AFM tip apex and the Apo-CaM molecule (FIG. 13A), and the typical CaM protein rupture event recorded by the force-time curve (FIG. 13B).

FIGS. 14A-14B: Comparison of AFM force curves measured under the condition of the non-activated CaM with a rupture event recorded (FIG. 14A), and the activated CaM with no rupture event observed (FIG. 14B).

FIGS. 15A-15D: Characterization of CaM rupture force and loading energy. FIG. 15A shows the Gaussian distribution of the rupture force of single Apo-CaM molecules under 1500 nm/s approaching velocity. FIG. 15B shows the estimated average force loading distance after adjustment with standard deviation, under the assumption that the atomic level contact between AFM tip and the Apo-CaM molecule occurs after the force reaches 5, 10, 15, 20, and 25pN, respectively, to counter the hydration shell. FIG. 15C shows the AFM electropiezo displacement distribution, which is defined as the distance traveled by AFM electropiezo from the start of the force loading on a targeted Apo-CaM molecule to the point of rupture. FIG. 15D shows the distribution of calculated loading energy under 1500 nm/s loading velocity.

FIGS. 16A-16B: AFM force curve of compressive force manipulation on a single tau protein molecule (FIG. 16A), and illustration of tau protein rupture under compressive force under K⁺ ion existence (FIG. 16B).

FIGS. 17A-17D: Characterization of tau protein rupture threshold force distograms measured under different compressive force loading rates: 3000 pN/s (FIG. 17A), 6000 pN/s (FIG. 17B), 12000 pN/s (FIG. 17C), and 60000 pN/s (FIG. 17D).

FIGS. 18A-18D: Tau protein rupture force threshold values measured under different force loading rates (FIG. 18A), tau protein rupture energy distributions under K⁺ existence (FIG. 18B), illustration of the stress vs. stain curves of a balloon model and a cotton-ball model (FIG. 18C), and a typical force curve measured from a tau protein under the compressive force loading with the existence of Mg²⁺ (FIG. 18D). No rupture event was observed.

FIGS. 19A-19B: 2D plot of force vs. displacement of pulling rupture events for identifying the tangled and aggregated tau-tau protein states. FIG. 19A shows the background signal distribution with only one tau protein involved. FIG. 19B shows the signal distribution of tau-tau tangled and aggregated protein under the force pulling measurement.

FIG. 20: Diagram of the AFM-FRET force manipulation microscopy instrument.

FIGS. 21A-21B: FIG. 21A shows the experimental setup of single-molecule AFM-FRET nanoscopy used in the examples herein. Specifically, FIG. 21A shows the schematic diagram of a coaxial laser and AFM tip. FIG. 21B shows an optical image of the laser focus spot under AFM tip scanning. The bright spot indicates the laser beam position.

FIGS. 22A-22B: Force mapping on a single protein molecule. FIG. 22A shows four possible positions of a protein molecule under the AFM tip and the different assessment of the acting force on the protein molecule. FIG. 22B represents the total number of ways a protein molecule can be located under the AFM tip. The green box represents where the protein molecule can be compressed without any trace of tensile force, whereas the blue box represents where the protein molecule can be compressed with a very small to negligible amount of tensile force.

FIG. 23: Procedures of AFM-focus-point alignment in the AFM-FRET correlated measurement.

FIG. 24: Different FRET intensity trajectories correlated with compressive force curve measurement.

FIG. 25: Specific single-molecule trajectory of the FRET correlation with the compressive force curve measurement, and the protein rupture event occurs at the time zero.

FIG. 26: One scanning period of the fluorescence intensity-time trajectory for the signal-to-noise ratio control experiment of the AFM-FRET nanoscopy setup.

FIG. 27: Force trajectory of single HPPK protein rupture plotted with AFM piezoelectric displacement.

FIGS. 28A-28B: Single CaM protein rupture data. FIG. 28A shows force vs AFM piezoelectric displacement data of a single CaM protein rupture. FIG. 28B shows a distribution of AFM piezoelectric displacement under 100 nm/s loading velocity.

FIG. 29: Schematic of tethering single protein molecules on a cover glass surface.

FIG. 30: Typical compressive force curves recorded under different conditions with and without Ca²⁺ for CaM at activated and non-activated states.

FIG. 31: Example of an experimental force curve on a single Apo-CaM molecule.

FIG. 32: Illustration of the protein entanglement process using hands for illustrative purposes. Two hands depict two tau protein molecules. Crowded proteins simultaneously rupture and then spontaneously refold to an entangled folding state, different from either folded or unfolded states of the tau protein, which may be a pathway for the tau protein aggregation that is related to a number of neurodegenerative diseases.

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

A protein under compressive force can act either as a “balloon” or a “cotton ball.” A balloon may rupture under compressive force and a cotton ball only changes shapes but does not rupture. By charge electric field modification, the protein rigidity can be softened to a more flexible form that can diffuse compressive forces by releasing stress to the local environment and through protein shape changes. In accordance with the present disclosure, disruption of a protein's three-dimensional structure can be caused by pico-Newton compressive forces, and this disruption can be stopped by micromolar or millimolar concentrations of cations, such as, but not limited to, Ca²⁺, Mg²⁺, or Zn²⁺. This provides for various avenues for investigation mechanisms and drug treatment, as well as treatments for conditions involving disrupted protein structure.

For example, provided herein are compositions useful for treating, preventing, or ameliorating a neurodegenerative diseases comprising a micromolar or millimolar concentration of a cation, such as Ca²⁺, Mg²⁺, Zn²⁺, or combinations thereof. Such compositions may further include one or more pharmaceutically acceptable carriers, diluents, or adjuvants.

Pharmaceutical compositions of the present disclosure may comprise an effective amount of a cation (an “active” ingredient), and/or additional agents, dissolved or dispersed in a pharmaceutically acceptable carrier. The preparation of a pharmaceutical composition that contains at least one compound or additional active ingredient will be known to those of skill in the art in light of the present disclosure, as exemplified by Remington's Pharmaceutical Sciences, 2003, incorporated herein by reference. Moreover, for animal (e.g., human) administration, it is understood that preparations should meet sterility, pyrogenicity, general safety, and purity standards as required by FDA Office of Biological Standards.

A composition disclosed herein may comprise different types of carriers depending on whether it is to be administered in solid, liquid or aerosol form, and whether it need to be sterile for such routes of administration as injection. Compositions disclosed herein can be administered intravenously, intradermally, transdermally, intrathecally, intraarterially, intraperitoneally, intranasally, intravaginally, intrarectally, intraosseously, periprosthetically, topically, intramuscularly, subcutaneously, mucosally, intraosseosly, periprosthetically, in utero, orally, topically, locally, via inhalation (e.g., aerosol inhalation), by injection, by infusion, by continuous infusion, by localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 2003, incorporated herein by reference).

The actual dosage amount of a composition disclosed herein administered to an animal or human patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein. Naturally, the amount of active ingredient(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In certain embodiments, a composition herein and/or additional agent is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsules, they may be compressed into tablets, or they may be incorporated directly with the food of the diet.

In further embodiments, a composition described herein may be administered via a parenteral route. As used herein, the term “parenteral” includes routes that bypass the alimentary tract. Specifically, the pharmaceutical compositions disclosed herein may be administered, for example but not limited to, intravenously, intradermally, intramuscularly, intraarterially, intrathecally, subcutaneous, or intraperitoneally (U.S. Pat. Nos. 6,753,514, 6,613,308, 5,466,468, 5,543,158; 5,641,515, and 5,399,363 are each specifically incorporated herein by reference in their entirety).

Solutions of the compositions disclosed herein as free bases or pharmacologically acceptable salts may be prepared in water suitably mixed with a surfactant, such as hydroxypropylcellulose. Dispersions may also be prepared in glycerol, liquid polyethylene glycols and mixtures thereof, and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In some cases, the form should be sterile and should be fluid to the extent that easy injectability exists. It should be stable under the conditions of manufacture and storage and should be preserved against the contaminating action of microorganisms, such as bacteria and fungi. The carrier can be a solvent or dispersion medium containing, for example, water, ethanol, polyol (i.e., glycerol, propylene glycol, liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be brought about by various antibacterial and antifungal agents, such as, but not limited to, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption such as, for example, aluminum monostearate or gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

Sterile injectable solutions are prepared by incorporating the compositions in the required amount in the appropriate solvent with various other ingredients enumerated above, as required, followed by filtered sterilization. Generally, dispersions are prepared by incorporating the various sterilized compositions into a sterile vehicle which contains the basic dispersion medium and the required other ingredients from those enumerated above. In the case of sterile powders for the preparation of sterile injectable solutions, some methods of preparation are vacuum-drying and freeze-drying techniques which yield a powder of the active ingredient plus any additional desired ingredient from a previously sterile-filtered solution thereof. A powdered composition is combined with a liquid carrier such as, but not limited to, water or a saline solution, with or without a stabilizing agent.

In other embodiments, the compositions may be formulated for administration via various miscellaneous routes, for example, topical (i.e., transdermal) administration, mucosal administration (intranasal, vaginal, etc.), and/or via inhalation.

Pharmaceutical compositions for topical administration may include the compositions formulated for a medicated application such as an ointment, paste, cream, or powder. Ointments include all oleaginous, adsorption, emulsion, and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones, and luarocapram. Possible bases for compositions for topical application include polyethylene glycol, lanolin, cold cream, and petrolatum, as well as any other suitable absorption, emulsion, or water-soluble ointment base. Topical preparations may also include emulsifiers, gelling agents, and antimicrobial preservatives as necessary to preserve the composition and provide for a homogenous mixture. Transdermal administration of the compositions may also comprise the use of a “patch.” For example, the patch may supply one or more compositions at a predetermined rate and in a continuous manner over a fixed period of time.

In certain embodiments, the compositions may be delivered by eye drops, intranasal sprays, inhalation, and/or other aerosol delivery vehicles. Methods for delivering compositions directly to the lungs via nasal aerosol sprays has been described in U.S. Pat. Nos. 5,756,353 and 5,804,212 (each specifically incorporated herein by reference in their entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts and could be employed to deliver the compositions described herein. Likewise, transmucosal drug delivery in the form of a polytetrafluoroetheylene support matrix is described in U.S. Pat. No. 5,780,045 (specifically incorporated herein by reference in its entirety), and could be employed to deliver the compositions described herein.

It is further envisioned the compositions disclosed herein may be delivered via an aerosol. The term aerosol refers to a colloidal system of finely divided solid or liquid particles dispersed in a liquefied or pressurized gas propellant. The typical aerosol for inhalation consists of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight, and the severity and response of the symptoms.

In particular embodiments, the compounds and compositions described herein are useful for treating, preventing, or ameliorating certain neurodegenerative diseases or conditions. Furthermore, the compounds and compositions herein can be used in combination therapies. That is, the compounds and compositions can be administered concurrently with, prior to, or subsequent to one or more other desired therapeutic or medical procedures or drugs. The particular combination of therapies and procedures in the combination regimen will take into account compatibility of the therapies and/or procedures and the desired therapeutic effect to be achieved. Combination therapies include sequential, simultaneous, and separate administration of the active compound in a way that the therapeutic effects of the first administered procedure or drug is not entirely disappeared when the subsequent procedure or drug is administered.

Further provided herein is a method of causing a protein rupture comprising applying a picoNewton compressive force to a protein sufficient to cause the protein to spontaneously rupture.

Further provided herein is a method of studying protein rupture involving the use of a customized atomic force microscope (AFM). The AFM is customized to substantially reduce the signal-to-noise level compared to a conventional AFM, and can be utilized to cause and monitor protein rupture by applying a picoNewton compressive force on a protein.

### EXAMPLES

An atomic force microscope is a useful tool to study enzymatic reactions, the structure of biomolecules, protein-protein interactions, and membrane dipole potential. Force manipulation, mainly using an atomic force microscope (AFM), has been extensively applied on interrogating protein structure and function. Typically, the mechanical force applied by an AFM tip on a protein molecule can be either compressive force or pulling force. Notably, pulling force spectroscopy has provided important knowledge about the mechanical, chemical, and structural properties of protein molecules such as folding/unfolding of biomolecules, peptide-peptide interactions, and molecular interactions. Nevertheless, it is also crucial to analyze the impact of compressive force on proteins in terms of their structures, associated functions, and activities. There are a number of technical approaches to combine single molecule spectroscopy and imaging technique with AFM-correlated microscopy. A combined AFM-TIRF microscopy technique which can probe unfolding dynamics of a small ubiquitin protein along the vertical axis has been developed. A “confocal laser scanning microscope/AFM system” has also been developed where one can simultaneously record the fluorescence spectra of a green fluorescence protein while applying mechanical force on it. Single-molecule optical detection, especially single-molecule fluorescence resonance energy transfer (smFRET), has been widely used to study biomolecules like DNA, RNA, and proteins. It provides detailed information of biomolecules' conformational changes in real time with sub-nanometer resolution. Furthermore, combining smFRET with AFM, an AFM-FRET spectroscopic nanoscope has been demonstrated to be a powerful approach for manipulating and exploring protein structure and functions.

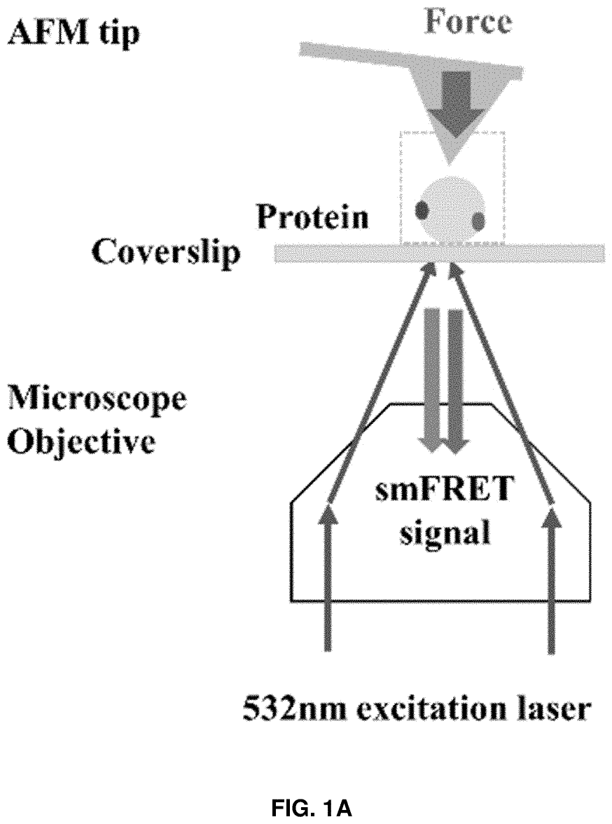

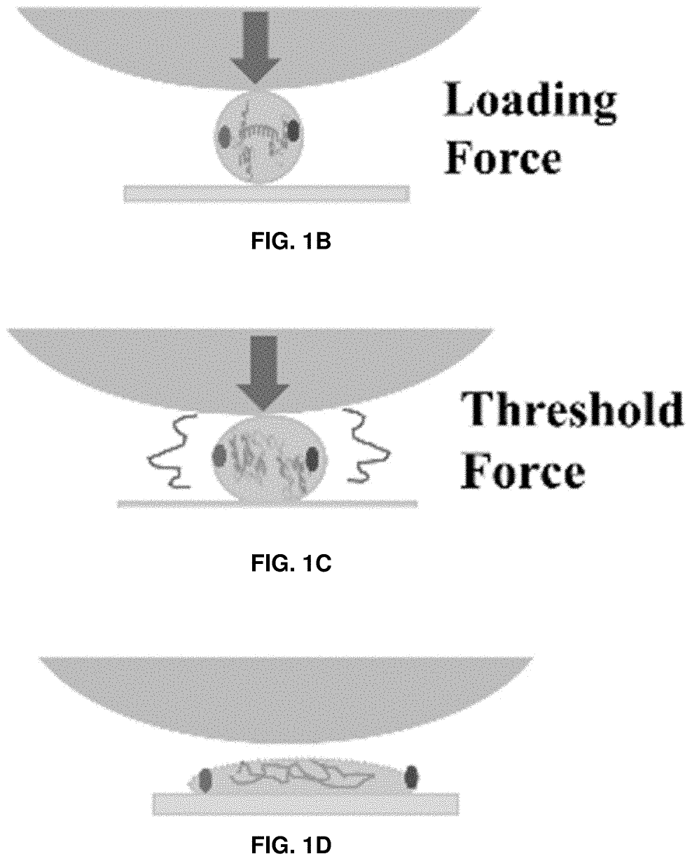

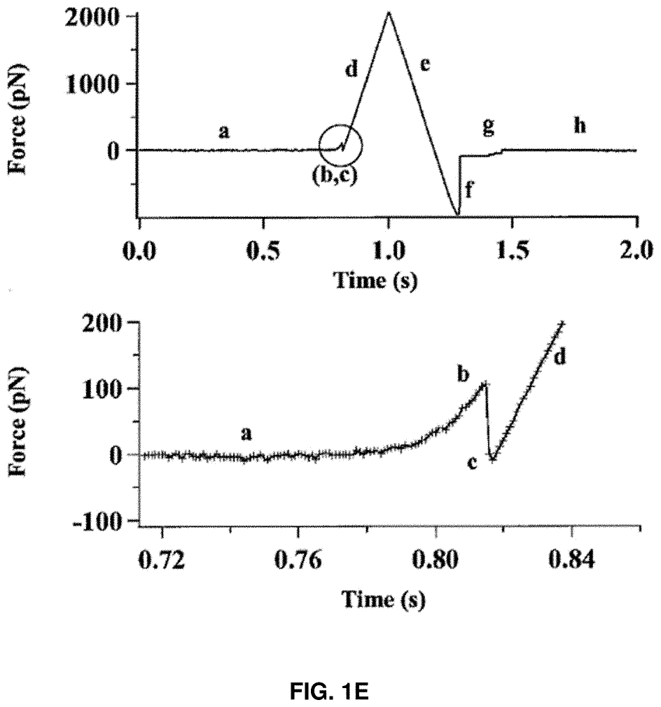

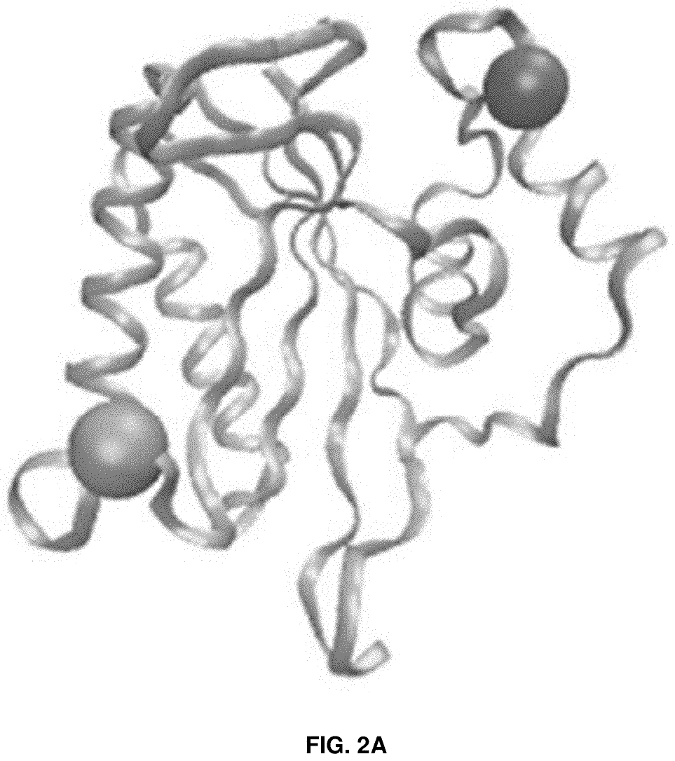

Using a single-molecule AFM-FRET spectroscopic nanoscope, pico-Newton level compressive force was applied on a targeted protein molecule, as described in these examples. Simultaneously, the conformational response of the individual protein was probed by both AFM force spectroscopy and smFRET spectroscopy (FIGS. 1A-1D). The experimental work (FIG. 1) on dissecting the protein response to compressive force applied by an AFM tip are described in these examples. It has been found that protein, an enzyme, and a signaling globular protein, undergo a process leading to a structural abrupt and spontaneous rupture (FIGS. 1B-1E) when the force reaches˜12-75 pN. Although a virus shell abrupt failure and structural change of a membrane protein under mechanical force applied by AFM has been reported previously, the phenomena of a protein structure abrupt rupture under a compressive force demonstrated in these examples represent previously unknown protein properties. The ruptured state of the protein can recover spontaneously through refolding or can be trapped at a disordered state for hundreds of milliseconds to seconds.

FIG. 1 shows the AFM-FRET nanoscopy setup and the schematic representation of a single protein molecule rupture. FIG. 1A shows AFM-FRET nanoscopy analysis and manipulation of protein rupture. Real-time force trajectories and correlated FRET trajectories are collected simultaneously in the experiment. The sample is under buffer solution. FIG. 1B shows a scheme of the interaction of the AFM tip apex with the HPPK molecule, showing the compressive force loading on the targeted protein. FIG. 1C shows the protein structure abruptly and spontaneously ruptures when the compressive force amplitude reaches a threshold value. FIG. 1D shows the loading force abruptly drops at the time of the protein structure rupture.

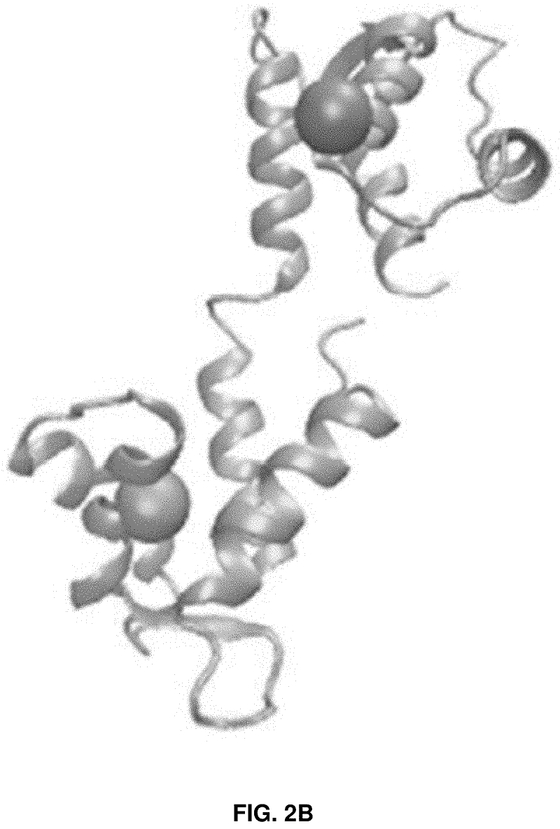

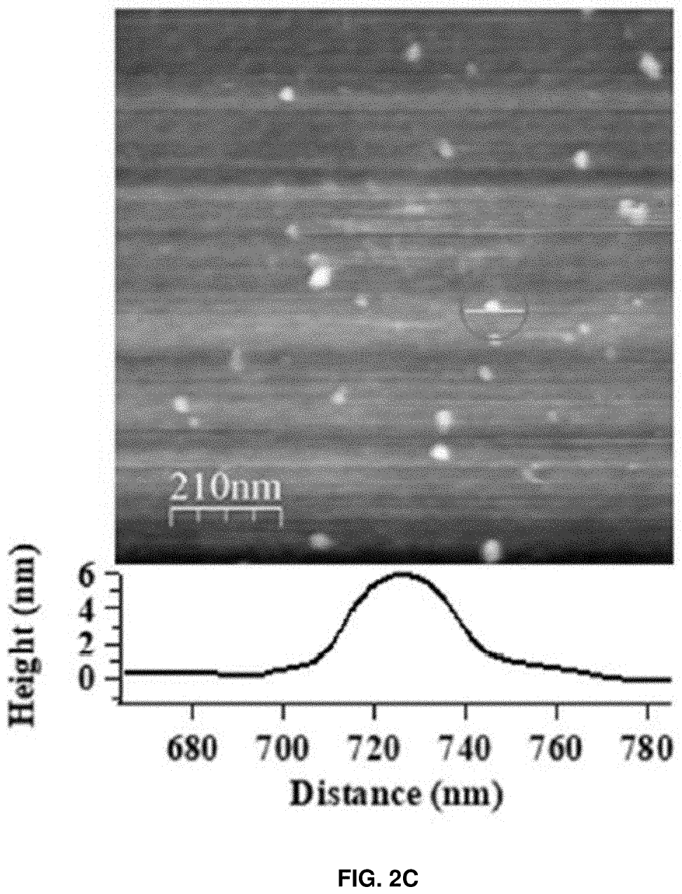

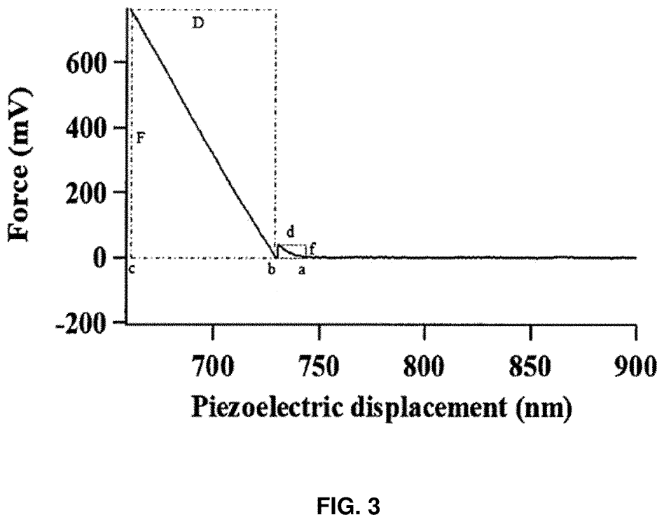

6-hydroxymethyl-7,8-dihydropterin pyrophosphokinase (HPPK) is a 158-residue monomer kinase enzyme protein which acts as a catalyst in the pyrophosphorylation reaction that drives the conversion of 6-hydroxymethyl-7,8-dihydropterin (HP) to 6-hydroxymethyl-7,8-dihydropterin pyrophosphate (HPPP) in the presence of ATP leading to the biosynthesis of folate cofactors. AFM was used to apply the compressive force on single protein molecules that are tethered on the glass cover slip surface at a controlled low density for single-molecule measurements. The enzyme HPPK was labeled with Cy3/Cy5 donor acceptor FRET pair on the amino acid residues 88 and 142, respectively (FIG. 2A), for probing the conformational changes of single protein under the compressive force.

FIG. 2A shows the structure of the HPPK molecule with FRET dye pair (Collected from Protein Data Bank, and processed with VMD software) Amino acid residues 88 and 142 were mutated with Cysteine and labeled with Cy3 and Cy5 dye, respectively. FIG. 2B shows the structure of CaM molecule with FRET dye pair Amino acid residues 34 and 110 were mutated with Cysteine and labeled with Cy3 and Cy5 dye, respectively. FIG. 2B shows the structure of the CaM molecule with FRET dye pair Amino acid residues 34 and 110 were mutated with Cysteine and labeled with Cy3 and Cy5 dye, respectively. FIG. 2C shows the AFM image of single HPPK molecules on a cover glass surface (256×256 points/line) and the topography of the encircled single HPPK protein molecule along the yellow line. The sample was prepared with ten times higher HPPK protein population as no optical measurement was involved. The height of the protein molecules on the cover glass was around 5 nm. The width measurement of the protein was affected by image artifacts due to the much larger size of the AFM tip. From the height profile, the radius of the AFM tip curvature was calculated. The radius of the AFM tip curvature was around 33 nm.

The structural rupture of a single HPPK protein under compressive force applied by AFM tip was evaluated. FIG. 1E shows the force response trajectory of the tip approaching an individual protein molecule attached on the coverslip. When the AFM tip touches a single protein molecule, a compressive force is applied on the targeted protein, the force monotonically increases up to the level of about 60 pN (FIG. 1E), and then the force drops to zero abruptly.

FIG. 1E shows as the AFM piezo approaches the surface, when the tip is in contact with the protein molecule on the glass surface, a compressive force loading process starts. As the piezo continues to approach toward the surface, the compressive force on the protein molecule increases; simultaneously, the protein starts to deform. When the force reaches a threshold value, the protein molecule suddenly ruptures, and the force on the tip releases to zero. As the AFM piezo continues to approach the glass coverslip surface, the tip on glass cannot move anymore, and as a result, piezo displacement causes tip bending. Therefore, from top to bottom, the cantilever bending distance=the piezo displacement D (nm); and the direct AFM instrumental electronic signal was measured and recorded for the loading force, F (mV). If the cantilever force constant k (pN/nm) is known, then, from the reading of measured f (mV), the protein rupture threshold force (pN)=f_{F}^{D}k can be calculated. And this force for protein rupture is significantly smaller than the force calculated based on the piezo displacement distance, where, the force=product of k times d. Because a part of the piezo displacement is contributed from the protein deformation under force, this does not contribute to the actual force loading.

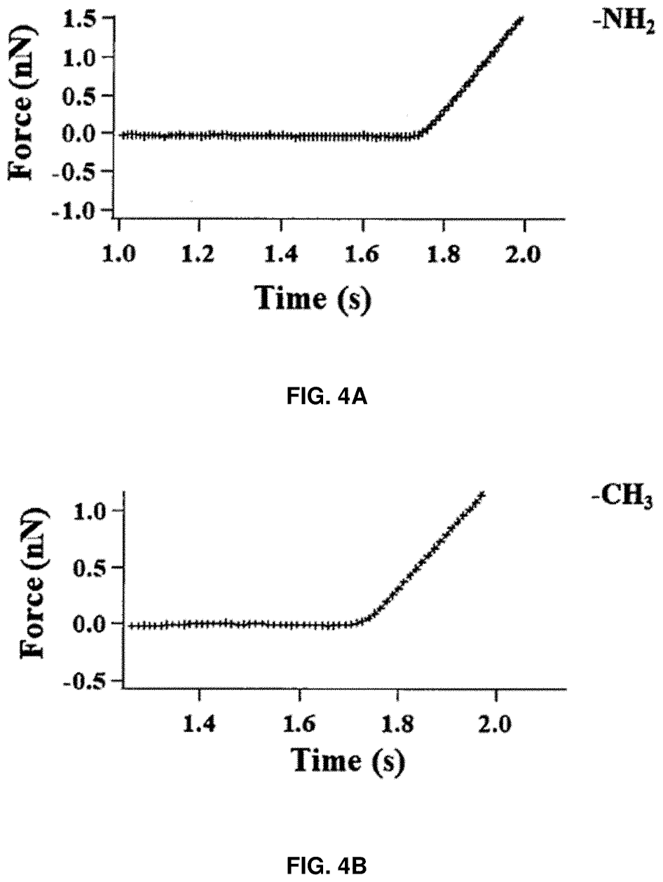

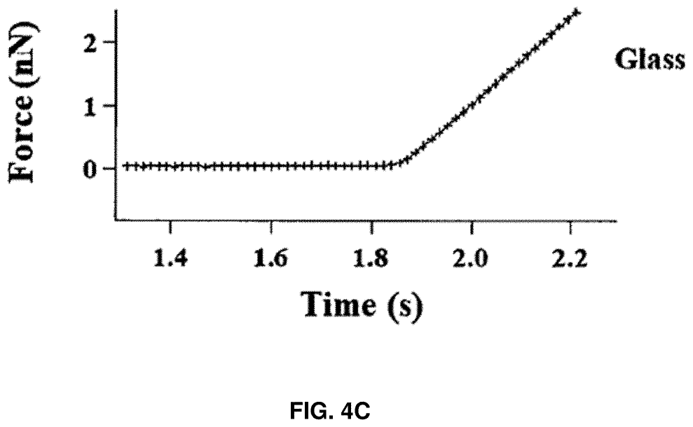

Control experiments show that there are no such force abrupt drop events recorded when the AFM tip approaches the modified surface of the cover glass with either (3-aminopropyl) trimethoxysilane or isobutyltrimethoxysilane with no protein molecules (FIG. 4).

To further prove that the force abrupt drop (47-75 pN drop) recorded in a force curve is due to the HPPK molecule sudden structural rupture, besides the simultaneous identification from the correlated recording of FRET trajectory, additional control experiments were performed on a glass surface that does not have a HPPK molecule tethered. All these experiments were done in three different conditions: non-modified bare glass surface, glass surface coated with only —NH₂ terminal ((3-aminopropyl) trimethoxysilane), and glass surface coated with only —CH₃ terminal (isobutyltrimethoxysilane). All the control experiments were conducted in PBS buffer solution (pH=7.4). The 47-75 pN abrupt force drop seen in the approaching force curves in the correlated measurements were not observed in the approaching force curves recorded in the blank control measurements under all of these three conditions. The results indicate that the pN level force abrupt drop events observed in the approaching curves are only associated with the targeted protein molecules, but not collapse or deconstruction of coated layer molecules. It is also consistent with the finding that the structural rupture under compressive force is a common behavior of protein molecules.

FIGS. 4A-4C show force curves on different glass surfaces where FIG. 4A shows glass coated with (3-aminopropyl) trimethoxysilane, FIG. 4B shows glass coated with isobutyltrimethoxysilane, and FIG. 4C shows non-modified bare glass.

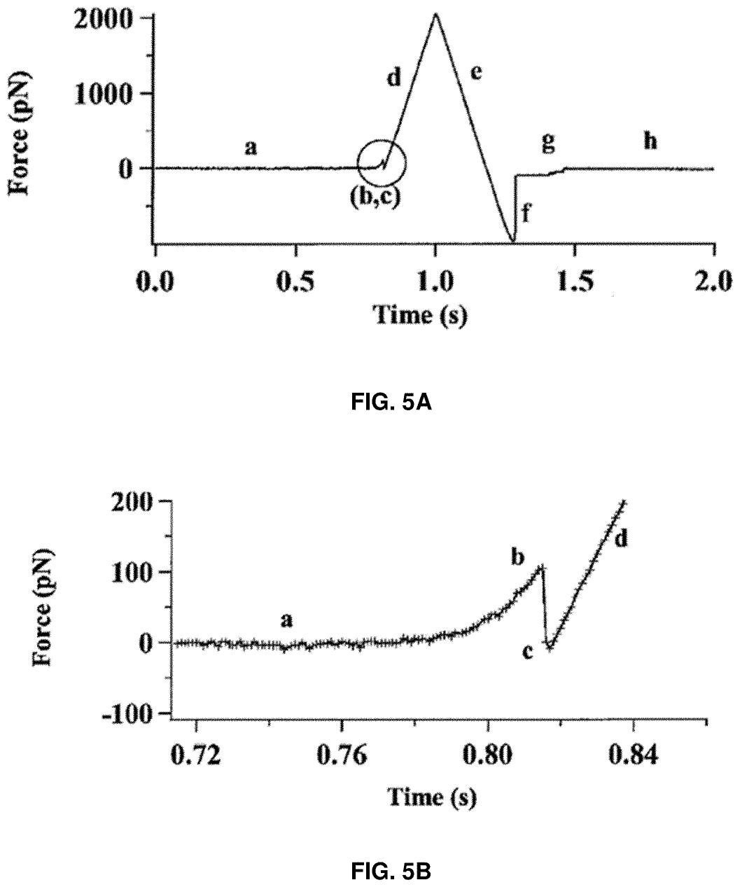

FIG. 5A shows the mechanical force curve of the AFM tip interaction with a single HPPK molecule. The HPPK protein rupture event was recorded in this curve. FIG. 5B shows a zoomed in view of the circled area of the force curve. Point a to point b (a→b) in FIGS. 1E, 5A-5B show the mechanical force engaging and loading on the targeted protein molecule; point b to point c (b→c) in FIGS. 1E, 5A-5B show the protein rupture event occurs when the loading force on the protein reaches the threshold value, resulting in an abrupt drop of the loaded mechanical force; point c to point d (c→d) in FIGS. 1E, 5A-5B show when the AFM tip touches the glass surface force loading resumes; point e to point h (e→h) in FIGS. 1E, 5A-5B show the following force curve represents routine and typical AFM tip retraction from the sample surface. Specifically, (e→f) force loading decreases as the AFM tip starts retracting from the glass surface. Negative force is due to the Van der Waals forces and electrostatic attraction between the AFM tip and glass surface. The force jumping back towards zero at point ‘f’ implies the separation of the tip from the surface. Point f to point g (f→g) in FIGS. 1E, 5A-5B show the AFM tip leaves the surface, but the protein is still attached to the tip, hence the relative force on the AFM tip changes with the different lobes of protein unfolding. Point g to point h (g→h) in FIGS. 1E, 5A-5B show a protein molecule being detached from the AFM tip.

Using the single-molecule AFM-FRET nanoscopic approach to manipulate the protein, detailed force-time trajectories and correlated smFRET-time trajectories were obtained.

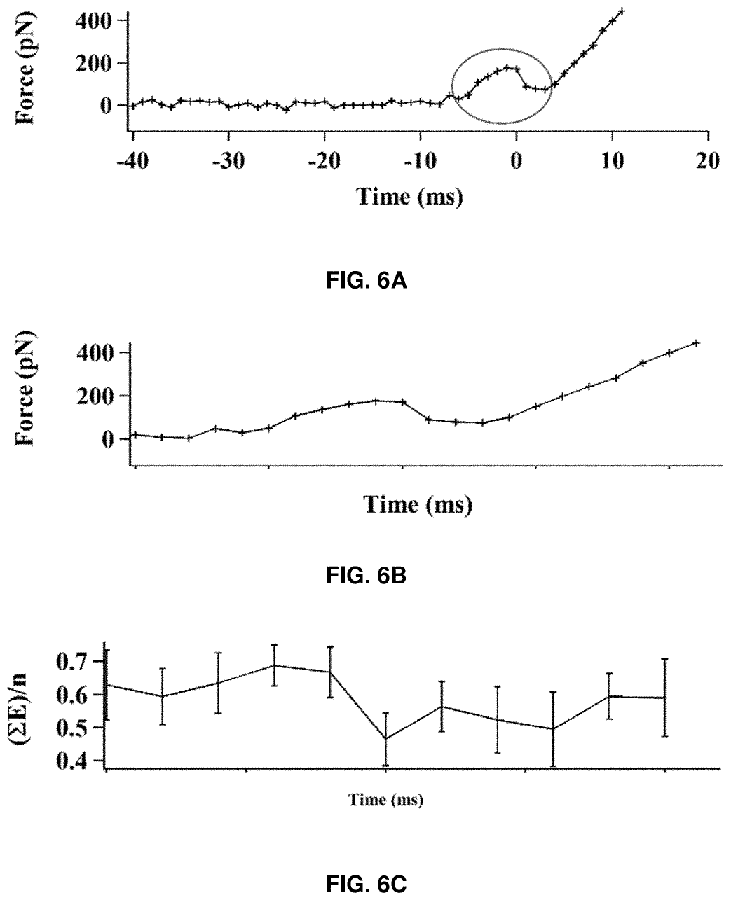

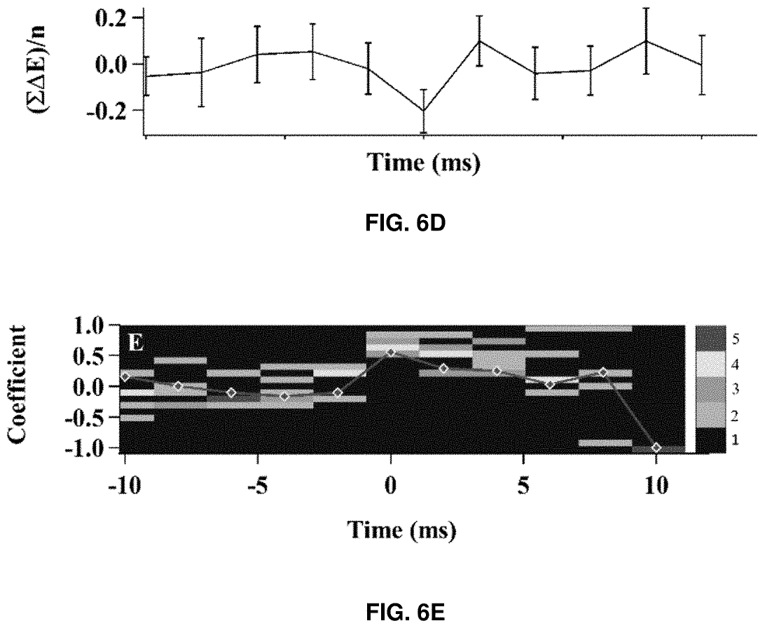

FIG. 6 shows correlated time trajectories of the AFM-FRET single-molecule protein force manipulation measurements. FIG. 6A shows the force trajectory of a pushing cycle. The time at the point of rupture considered as time zero, the time before rupture is (−)ve time, and the time after rupture is (+)ve time. FIG. 6B shows the force trajectory (at the zoomed in area of FIG. 6A) correlated with FRET efficiency trajectories. FIG. 6B shows a typical set of force-time trajectory correlated with smFRET-time trajectories. It is observed that FRET efficiency decreases at the same time when the force abruptly drops, indicating that the HPPK molecule experiences a sudden large conformational change under compressive force manipulation (FIG. 6C). Plotting average FRET efficiency changes (FIG. 6D), it is shown that there is a negative FRET efficiency change at the time when the protein rupture events occurred. Collectively, the correlated AFM-FRET single-molecule trajectories indicate that there is a significant change in the protein conformation at that point of the force drop (FIGS. 6B, 6C, and 6D). The sudden decrease of FRET efficiency reflects the sudden increase in distance between two labeled domains of the HPPK molecule, which represents a characteristic protein structure rupture event. The sudden conformational change probed by FRET coincides with the abrupt drop of the compressive force built up on the AFM cantilever. Based on these experimental results, the sudden structural rupture of the HPPK protein molecule was triggered by the compressive force, and the rupture event is spontaneous when the compressive force reaches the threshold value. Remarkably, it was observed that the protein structural abrupt rupture events occurred at a threshold amplitude from more than 90% of the single HPPK molecules examined.

FIG. 6C shows the average FRET efficiency of ten correlated trajectories with standard deviation error bar and binning time of 2 ms. FIG. 6D shows the average of FRET efficiency change with standard deviation error bar and binning time of 2 ms. FIG. 6E shows the distribution of Pearson's product-moment correlation coefficient r_{F,E} (T, n) of force and FRET efficiency near the moment of the protein rupture calculated from 13 sets of AFM-FRET correlated trajectories with 2 ms binning time. The red curve is the correlation coefficient trajectory calculated from the trajectory ‘A’ and its respective FRET efficiency trajectory. FIGS. 6B, 6C, 6D, and 6E share the same time axis.

To further characterize the HPPK structural rupture events, the Pearson's product-moment correlation coefficient, r_{F,E}, between FRET efficiency and force at the force abrupt drop time was analyzed, representing total correlation at r_{F,E}=1, and no correlation at r_{F,E}=0. Pearson's product-moment correlation coefficient analysis between FRET efficiency E(t) and measured force F(t) (eqn. 1) was used.

$${r}_{F , E} ( T , n ) = \frac{1}{n - 1} {\sum }_{t = T - ( n - 1 ) \cdot 2 ms}^{t = T} ( \frac{F ( t ) - \overline{F} ( t )}{{s}_{F} ( T )} ) ( \frac{E ( t ) - \overline{E} ( T )}{{s}_{E} ( T )} ) \qquad ( 1 )$$

Where T is the index of FRET efficiency time trajectory based on 2 ms bin; n is the number of data points within each calculation window (12 data points were used in the calculation (FIG. 6E), starting from 10 ms time bin before the rupture event and continues to 10 ms time bins after the rupture event, where, rupture event occurs at t=0. F(t) is force measured); E(t) is FRET efficiency calculated based on the smFRET measurements; F̅(T), Ē(T) are the sample averages within each calculation window; s_{F}(T) and s_{E}(T) are the sample standard deviations within each calculation window, which is defined as (eqn. 2)

$${s}_{x ( T , n )} = \sqrt{\frac{1}{n - 1} {\sum }_{t = T - ( n - 1 ) \cdot 2 ms}^{t = T} {( x ( t ) - \overline{x} ( T ) )}^{2}} \qquad ( 2 )$$

FIG. 6E shows that the Pearson's product-moment correlation coefficient reaches the maximum (close to 1) at the moment of the rupture events, which indicates higher correlation. The distribution of the coefficients is narrowly distributed at the time of rupture, indicating a stronger temporal correlation between the force drop and the protein rupture; whereas, the distribution is much broader away from the rupture event time (FIG. 6E). Although each rupture event can have a different force trajectory in terms of rupture threshold force value and force loading time, the release of compressive force and sudden decrease of FRET efficiency are highly correlated statistically.

Similarly, to confirm CaM protein rupture events, the distribution of Pearson's product-moment correlation coefficient between FRET efficiency and the force near the force abrupt drop time was plotted, showing that the Pearson's product-moment correlation coefficient reaches the maximum at the moment of the rupture events. The distribution of the coefficient is narrowly pin-pointed at the rupture event time, whereas the distribution tends to be much broader when t<−0.1 s and t>0.1 s away from the rupture event time.

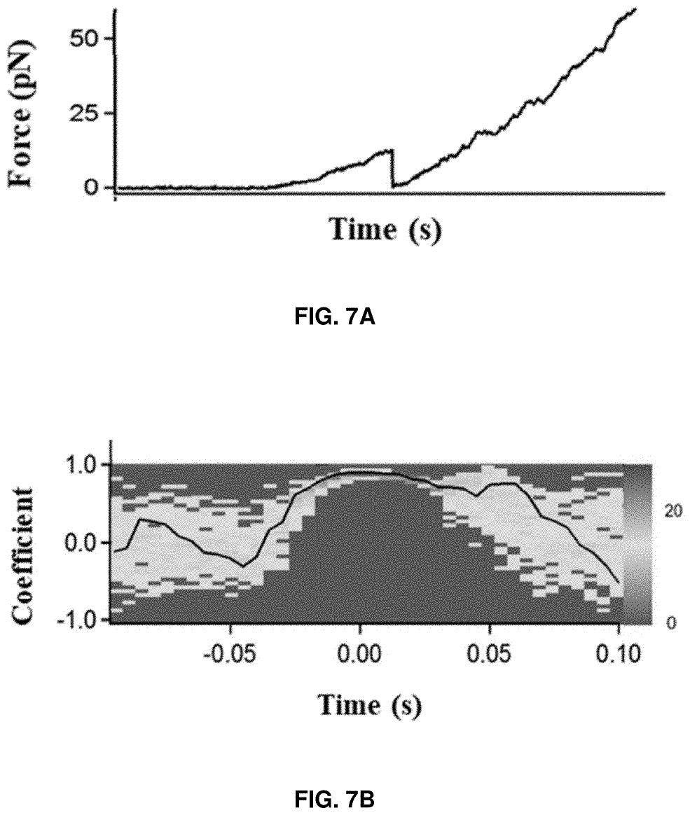

FIG. 7A shows the force trajectory of a pushing cycle on a single CaM molecule. Time at the point of rupture considered as time zero, time before rupture is (−)ve time, and time after rupture is (+)ve time. FIG. 7B shows the distribution of Pearson's product-moment correlation coefficient r_{F,E} (T, n) of force and FRET efficiency near the moment of the CaM protein rupture calculated from 68 sets of AFM-FRET correlated trajectories with 10 ms of binning time. The moment of rupture is redefined as time zero for the trajectories. The black curve is the correlation coefficient trajectory calculated from the trajectory ‘A’ and its respective FRET efficiency trajectory.

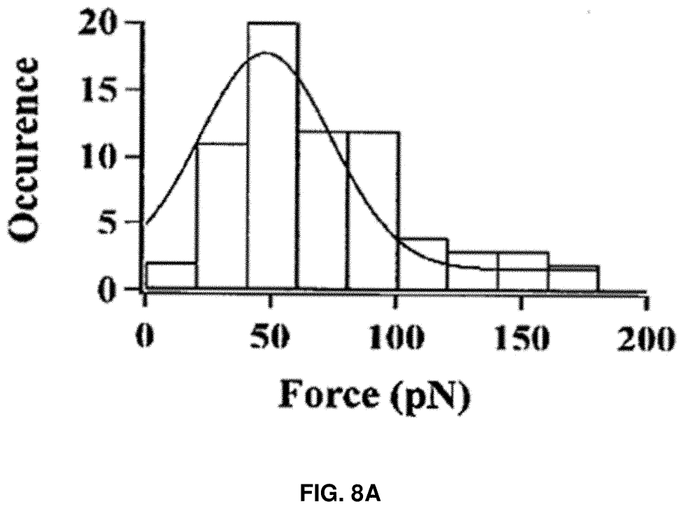

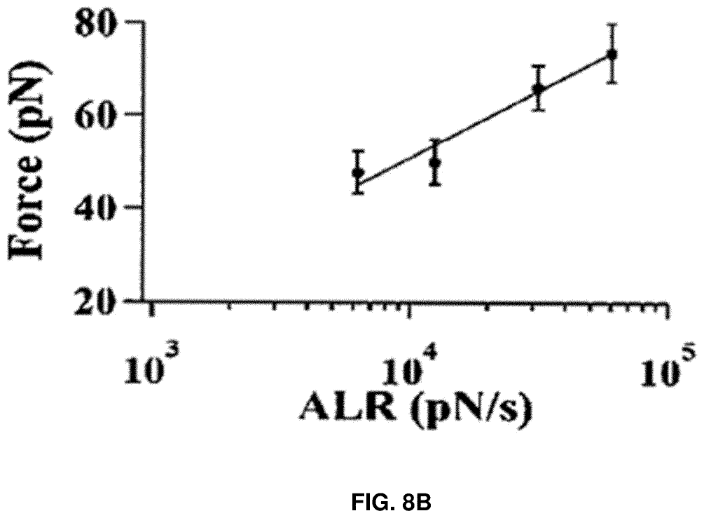

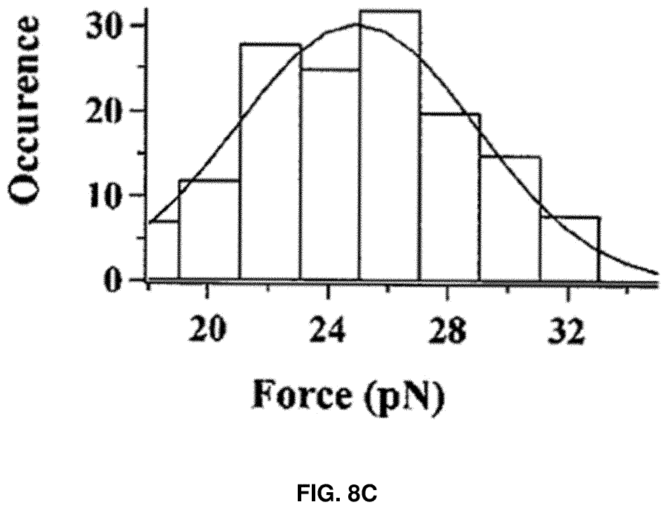

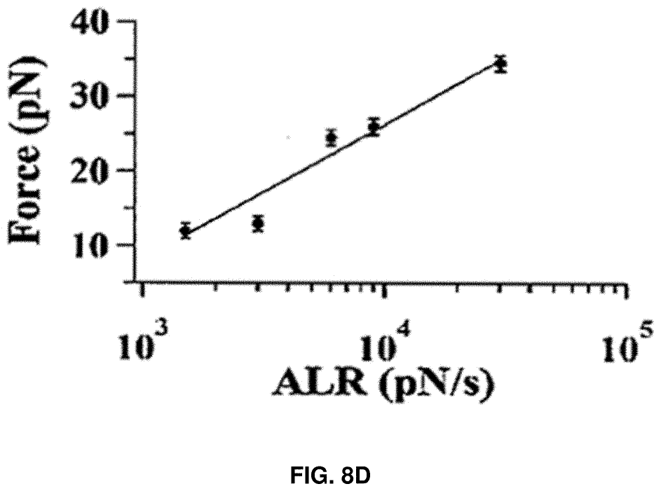

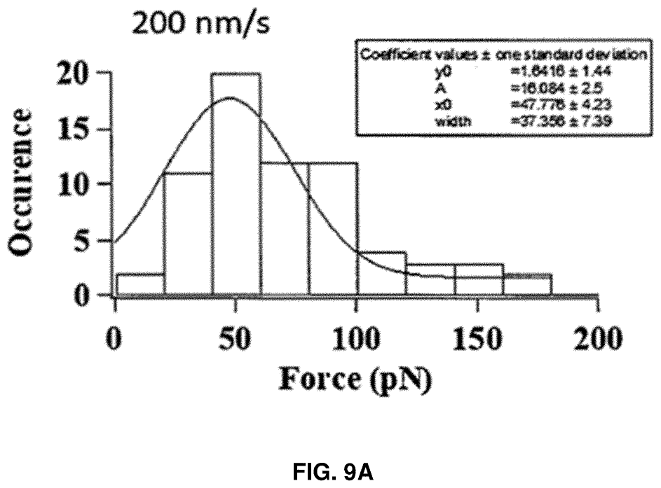

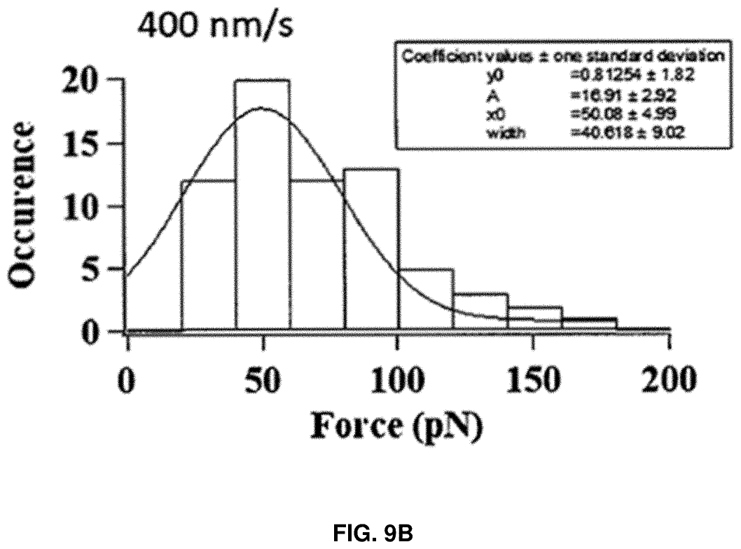

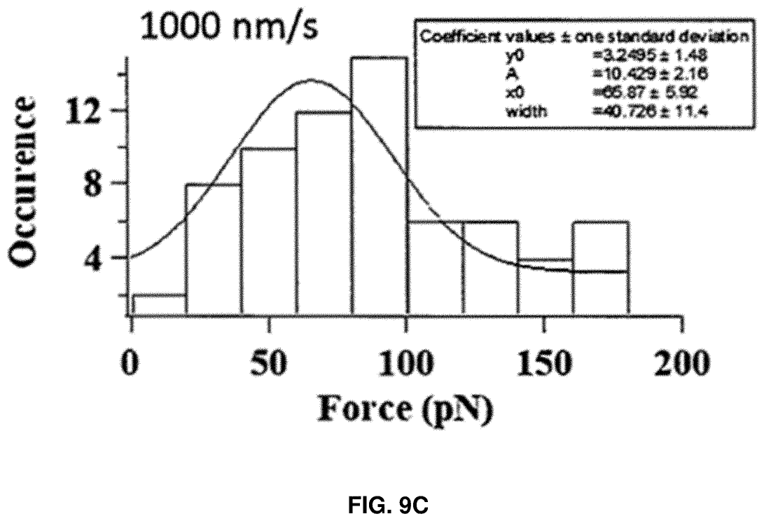

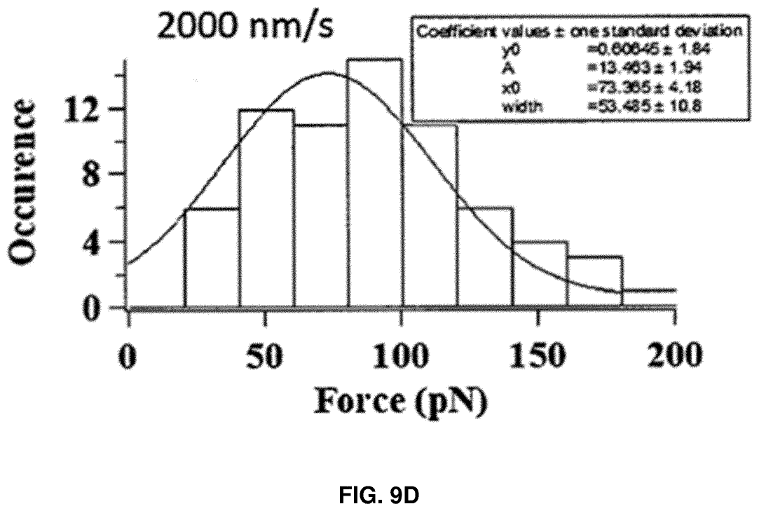

To evaluate the relation between the compressive threshold force and the tip approaching velocity, the experiment was repeated under different approaching velocities (FIGS. 8-9). The threshold force for a single protein HPPK rupture increases as the apparent loading rate (ALR) by the AFM tip increases (FIG. 8B). ALR equals the approaching velocity times the force constant of AFM cantilever. This dependence of the rupture threshold force upon the loading rate indicates that the protein rupture events and the compressive force loading process have the essential characteristics of two-state conformational transition under an external force. The results indicate that this process has an energy profile similar to a classical two state transition with an activation barrier.

FIGS. 8A-8D show the comparison of rupture force for different protein molecules, HPPK and CaM proteins. FIG. 8A shows the HPPK rupture threshold force distribution under 200 nm/s loading velocities. FIG. 8B shows the ALR dependent rupture threshold force distribution for HPPK. FIG. 8C shows the CaM rupture threshold force distribution under 200 nm/s loading velocities. FIG. 8D shows the ALR dependent rupture threshold force distribution for CaM. The unit of ALR is pN/s.

A two-state theoretical model, the Bell-Evans theory (eqn. 3), was used to analyze the linear dependence of rupture force with the logarithm of loading rate (FIG. 8B):

$$F ( r ) = ( \frac{{k}_{B} T}{{x}_{β}} ) ln \frac{{rx}_{β}}{{k}_{0} {k}_{B} T} \qquad ( 3 )$$

where F(r) is the most probable rupture force; kB, the Boltzmann's constant; T, temperature in Kelvin; X_{β} is the distance between the bonded state and the transition state where the activation barrier is located; r, the loading rate; and kₒ the off-rate constant at zero force. The fitting gives two model parameters, kinetic off rate constant kₒ=12.2±4.5 s⁻¹ and the distance from the bound to transition state, X_{β}=0.34±0.05 nm. Using kₒ, the barrier height of the transition state (ΔG) was also calculated using the following equation (eqn. 4), where h is the Planck's constant. ΔG of HPPK protein rupture is 27.0±9.8 k_{B}T.

$$- Δ G = {k}_{B} T ln ( \frac{{k}_{0} h}{{k}_{B} T} ) \qquad ( 4 )$$

To demonstrate the protein rupture behavior generally exists in other proteins, similar AFM force manipulations were carried out on individual calmodulin (CaM) proteins, and similar spontaneous ruptures of CaM under the pN force manipulation were observed. FIGS. 8A and 8C show that rupture force is consistently lower for the CaM molecule than that of HPPK. The mean rupture force from the distributions are 25 pN and 47 pN for CaM and HPPK molecules, respectively. ΔG was also calculated for CaM rupture, which is about 25.6±7.3 kBT using Bell-Evans Model. The characteristic behavior is due to different structural rigidity and a different vector orientation of the mechanical force inside different protein molecules. Nevertheless, the control experiment of CaM protein rupture under mechanical pN force manipulation shows that protein rupture behavior is not only HPPK-protein specific but a generally existant protein property and behavior, and the threshold rupture force value is protein specific.

To evaluate the relation between threshold force of protein rupture and AFM tip approaching velocity, i.e., the apparent loading rates (ALR), the experiments were repeated under four different ALRs. FIGS. 9A-9D show the statistical distribution of the threshold force at four different ALRs.

The threshold force required for a single protein HPPK to rupture increases from 47 pN to 75 pN as the approaching velocity increases from 200 nm/s to 2000 nm/s, and the threshold force increases linearly along with log (ALR) (FIG. 8B). This dependence of the threshold force upon the loading rate indicates that the protein rupture events and the compressive force loading process have the similar characteristics with a system of two-state conformational transition under an external force.

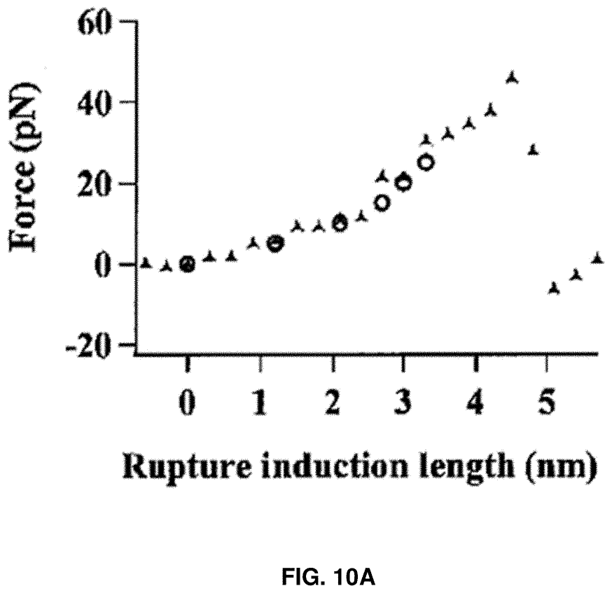

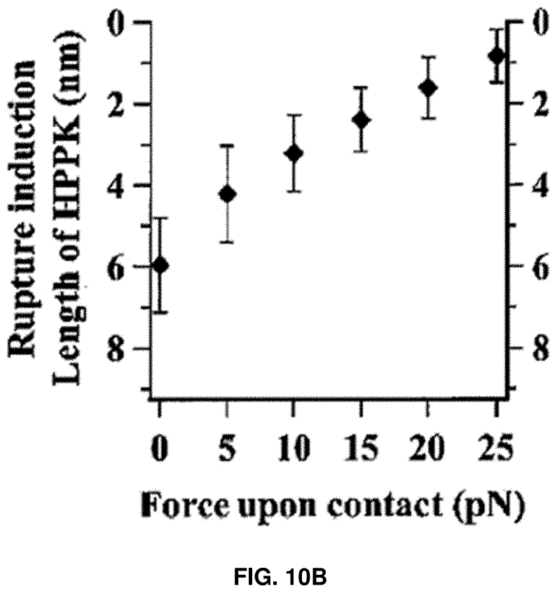

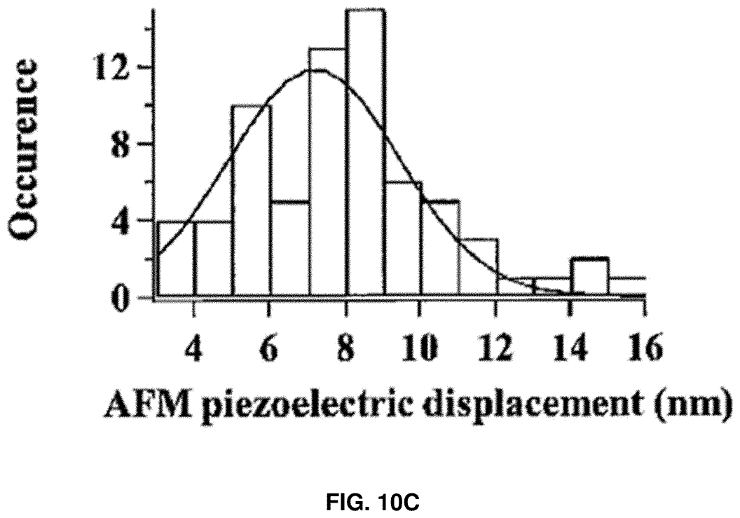

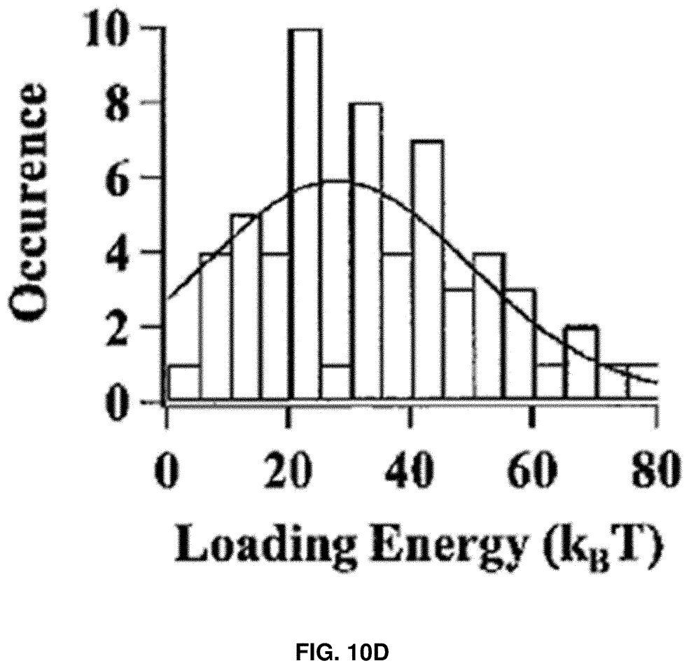

The real rupture induction length of a protein under compressive force was estimated. The length reflects the actual structural change experienced by the protein molecule at the process of the compressive force loading to the threshold value on an examined protein molecule. The force trajectories were further analyzed based on the following experimental facts: (1) the distance traveled by the AFM piezoelectric scanner exceeds the distance traveled by the AFM tip apex due to bending of the cantilever upon force loading, and the latter can be accounted by using the average cantilever force constant of 30 pN/nm; and (2) the aqueous solvation layer or the hydration shell around a protein is at least 1˜2 nm. Therefore, the apparent height of tethered protein molecule on the glass surface is at least few nm higher than what the crystal structure shows just considering the contribution of hydration shell. This attribution is further supported by the results of the force curve measurements. Notably, in all the force curves (FIG. 10A), the force increases much slower at the beginning of the force loading compared to the latter part of the force loading. It can be understood that the AFM tip apex interacts with the hydration shell first and only then touches the protein molecule surface after the initial force loading. Although it is difficult to precisely identify when the atomic level contact with the protein surface occurs in the actual measurements, it is estimated that the contact occurs after a certain level of force is reached in order to counter the water solvation layer around the targeted protein. Different assessments were plotted in FIG. 5B from a chosen “force upon contact” at 0, 5, 10, 15, 20, and 25 pN.

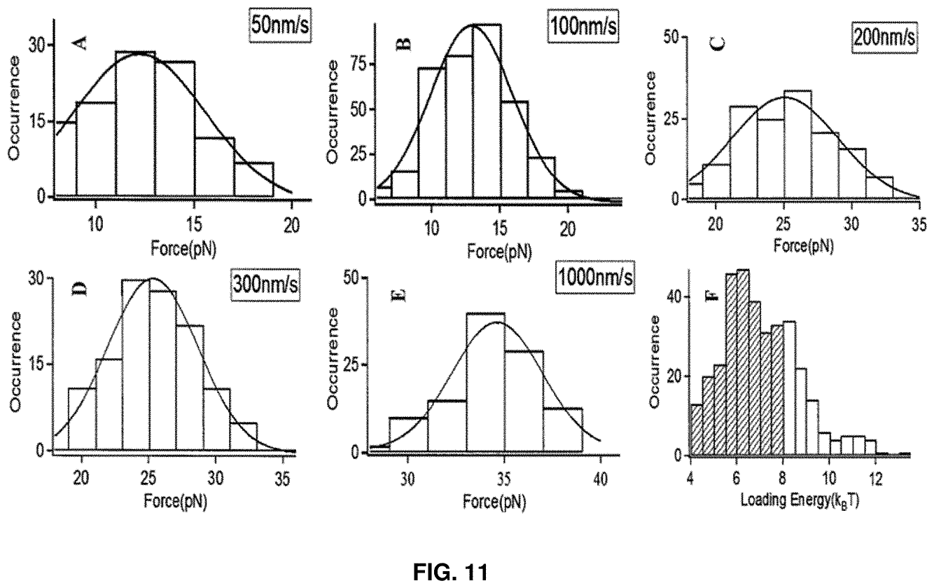

The AFM piezoelectric displacement distribution (FIG. 10C) is directly read out from the force curves. The displacements are measured from the moment when the force just starts to increase until the moment rupture event occurs. On average, the rupture events occurred at 5.0˜6.0 nm after the tip starts to load force upon protein molecule (FIG. 10C). The threshold compressive force loading energy, E_{loading}=0.5×threshold force×tip loading distance, was also estimated in the experiments (FIG. 10D), which is around 30 k_{B}T. Notably, for CaM molecules, the loading energy was around 4-8 k_{B}T (FIG. 11F), which is in the range of thermal energy fluctuation in living cells under 37° C., indicating that the observed protein rupture can be a common phenomenon inside the living cell. Although the loading energy for HPPK was not necessarily attainable by thermal fluctuation, such small amounts of energy could also be attained by other means and mechanisms inside a living cell.

FIGS. 10A-10D show the characterization of the compressive force loading on single HPPK molecules. FIG. 10A shows an example of experimental force curve. The small red circles show the point where the compressive force just exceeds 0 pN, reaches 5, 10, 15, 20, and 25 pN, respectively. FIG. 10B shows the estimated average rupture induction length after adjustment with standard deviation, assuming the atomic level contact between AFM tip and protein occurs after the force reaches 5, 10, 15, 20, and 25 pN, respectively. FIG. 10C shows AFM piezoelectric displacement distribution, which is defined as the distance traveled by AFM piezoelectric between the start of the force loading on a targeted protein and the protein rupture. The actual distance of force loading up to a threshold compressive force for inducing the protein rupture event can be significantly shorter due to the piezoelectric displacement calibration and the protein hydration shell. FIG. 10D shows the distribution of calculated loading energy under 200 nm/s loading velocity.

The experiments for CaM protein were repeated under five different ALRs. FIGS. 11A-11E show the statistical distribution of the threshold force at five different ALRs and characterization of CaM rupture force and loading energy. FIGS. 11A-11E show the rupture threshold force distribution in different loading velocities. FIG. 11F shows the distribution of calculated loading energy. Loading velocity is 100 nm/s. The threshold force required for a single CaM protein molecule to rupture increases from 12 pN to 35 pN as the approaching velocity increases from 50 nm/s to 1000 nm/s, and the threshold force increases linearly along with the log of ALR (FIG. 8D).

The findings involving calmodulin were further explored. Calmodulin is a ubiquitous calcium binding protein with 148 residues (16.7 KDa) that plays crucial roles in its Ca²⁺-ligated activated form in the transduction of Ca²⁺ signals. It performs this role by binding to several targets inside the cell including ion channels and a large number of enzymes and proteins.

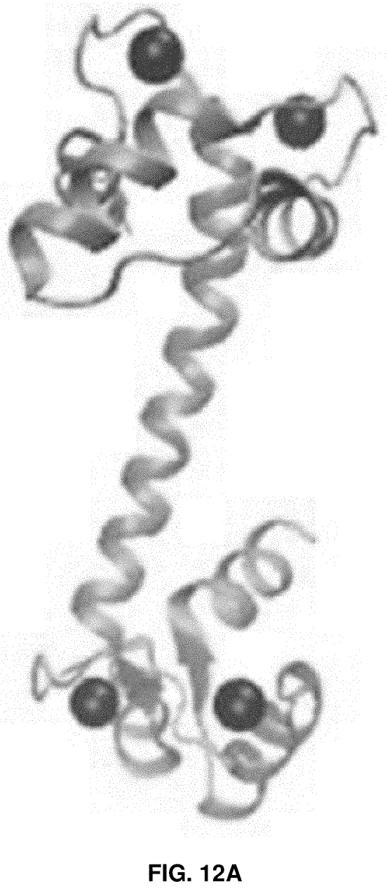

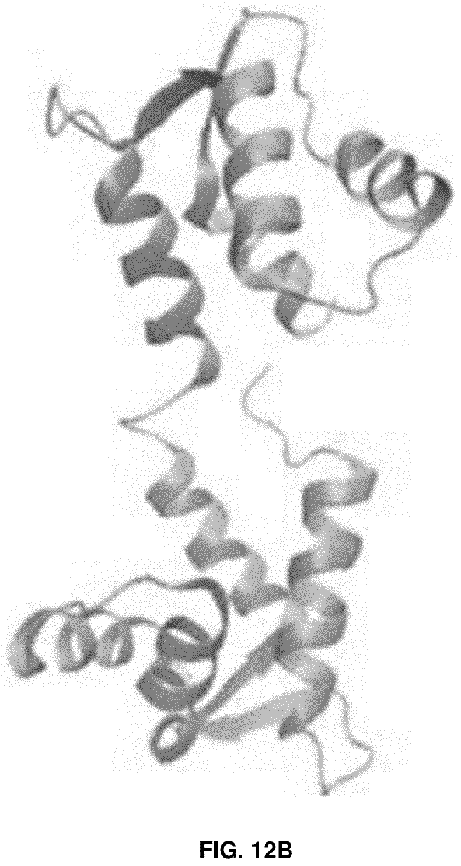

The crystal structure of Ca²⁺-ligated CaM has a very distinct dumbbell shape, where two approximately symmetrical globular C- and N-terminal domains are separated by a 27 residue long α-helical linker (FIG. 12A). Both globular domains contain two EF-hand motifs, and each of these motifs binds with one Ca²⁺ ion to sense intracellular calcium level. This elongated dumbbell conformation of Ca²⁺-ligated calmodulin exposes two hydrophobic patches centered on the concave surface of each lobe, which help the molecule to bind with ligands and activate a range of kinases. FIG. 12A shows the crystal structure of Ca²⁺-ligated calmodulin. FIG. 12B shows the NMR structure of Apo-calmodulin in solution.

When Ca²⁺ ions are removed from the EF hand motifs of calmodulin, it transforms to a more bound conformational state Apo-CaM from its prominent dumbbell shape (FIG. 12B). It is clear from the NMR study that both lobes of Apo-CaM form a globular four helix-bundle conformation. In this closed conformation hydrophobic residues are inaccessible to external ligands.

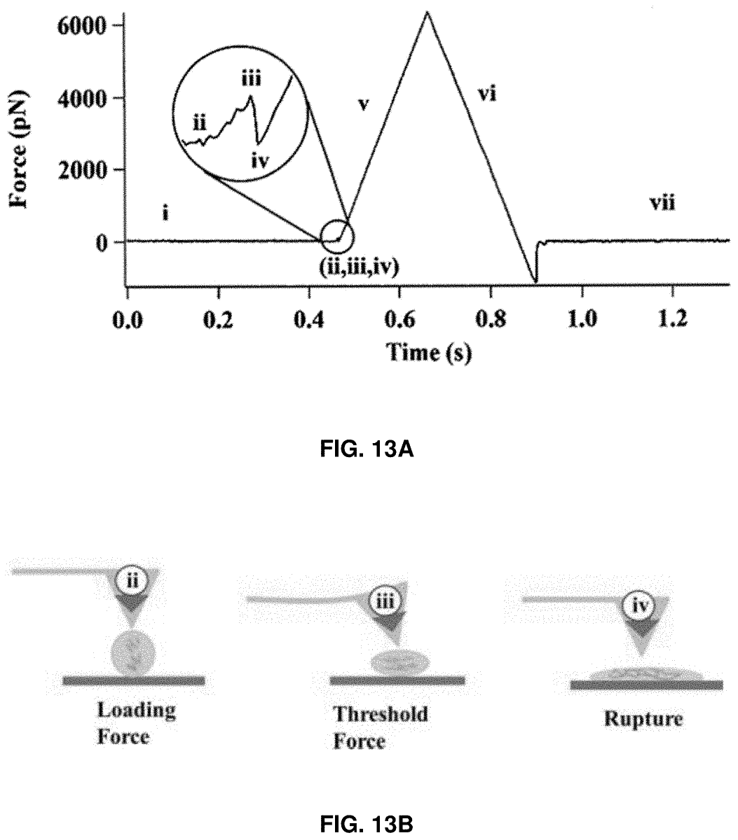

In the experiment of compressive force manipulation on both Apo- and Ca²⁺-ligated CaM, a modified AFM apparatus with an ultra-soft AFM tip was used to apply compressive force on a single protein molecule tethered to a glass coverslip through covalent bonding between the linker molecule and amino group of the protein, and its response was studied. It was found that Apo-CaM molecules undergo an abrupt spontaneous rupture at ˜70 pN (see supplemental information below) of compressive force. FIG. 13 shows a general pattern of force curve corresponding to a single Apo-CaM protein rupture and a cartoon scheme of AFM tip-protein interaction in the process of force loading on a protein molecule. It was also observed that force loading on a single Ca²⁺-ligated CaM molecule does not go through any such rupture event.

FIG. 13A shows the mechanical force curve of AFM tip interaction with a single Apo-CaM molecule. A rupture event was recorded in this curve. (i→ii) shows the AFM tip approaches to the single Apo-CaM molecule. At the point ‘ii’, the mechanical force loading starts as the AFM tip touches the surface of the targeted Apo-CaM molecule. (ii→iii) shows the force loading continues to a certain threshold value. At point ‘iii’ the loading force on the protein reaches the threshold value and the protein cannot hold the force anymore and gets spontaneously ruptured, resulting in the loaded mechanical force abruptly dropping to ‘iv’. (iv→v) shows the force loading resumes when the AFM tip touches the coverslip glass surface. (vi→vii) shows the part of the force curve representing typical AFM tip pulling up from the sample surface. FIG. 13B shows a scheme of the interaction of AFM tip apex and the Apo-CaM molecule. ‘ii’ represents the point where the compressive force loading starts on the protein. ‘iii’ represents the point where the force loading reaches a threshold value and the protein structure spontaneously ruptures. ‘iv’ represents the point where the loading force abruptly drops due to protein rupture.

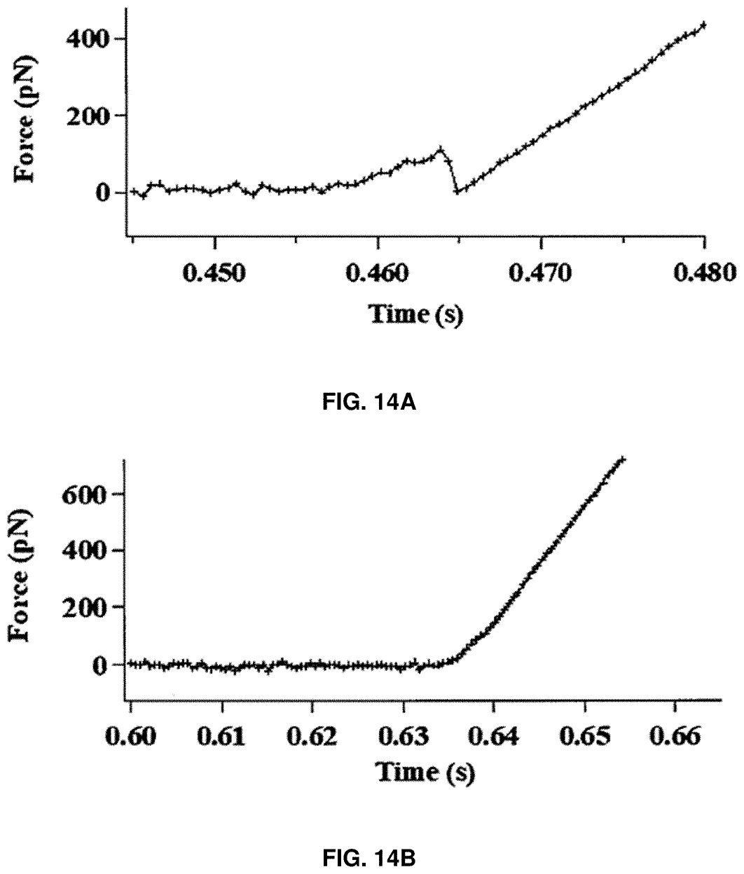

FIG. 14A shows a typical AFM force curve where an AFM tip approaches a single Apo-CaM molecule. The AFM apparatus has sufficient sensitivity and signal-to-noise ratio to record the entire pN level of force loading on a single protein molecule. Intramolecular hydrogen bonding, inter-domain friction force, liquid friction force with the solvent molecules, and intermolecular hydrogen bonding between protein and the solvent molecules contributes to the rigidity and enable the molecule to withstand pN amount of force. Significantly, an abrupt force drop was observed when the AFM force loading on the single Apo-CaM molecule reached a certain threshold value. This abrupt drop in the force curve corresponds to the sudden release of force on the AFM tip, i.e., the protein molecule can no longer hold the force at the threshold value, which causes a simultaneous and spontaneous collapse of a significant amount of intramolecular interactions and hydrogen bonds that hold the molecule together. This type of protein rupture events under compressive force have never been reported previously. Nevertheless, this protein property is highly significant and closely related to protein functions in living cells, as the thermal fluctuations of local force may provide such pN force fluctuations and trigger protein structure collapse, a catastrophic unfolding event, that may be associated with protein dysfunction, aggregation, and misfolding.

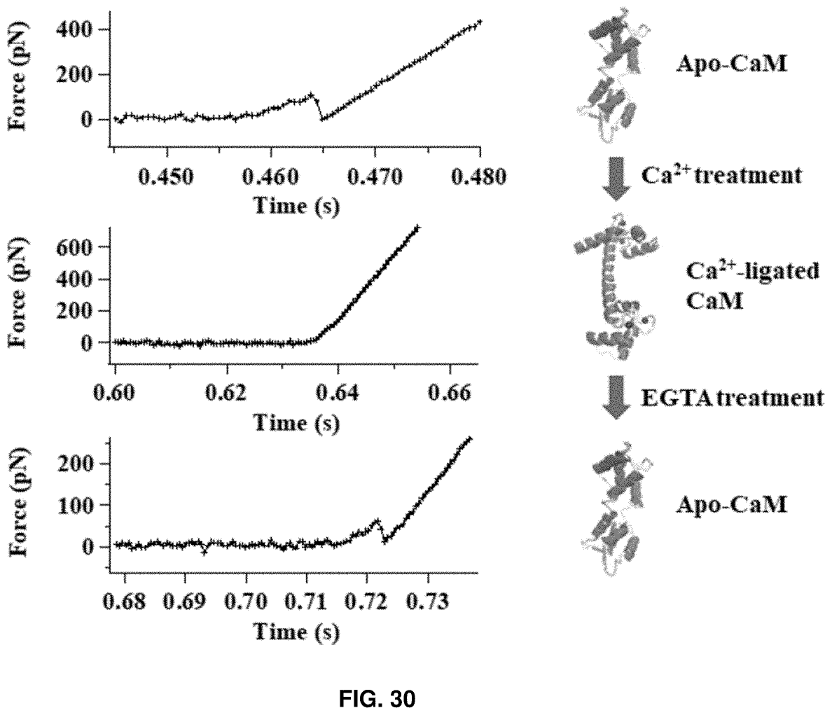

The same experiment was carried out on Ca²⁺-ligated CaM. FIG. 14B represents the typical AFM compressive force curve on a Ca²⁺-ligated single CaM molecule. There is no abrupt force drop in this force curve, which signifies the absence of rupture event under compressive force. It was also noticed that after treatment with EGTA, which takes away Ca²⁺ ions from Calmodulin converting it to Apo-CaM, the rupture event was recovered (FIG. 30). Nevertheless, repeated rupture events on a single protein molecule have been noticed when a repeated force manipulation experiment on the same protein molecule has been carried out. This indicates that the ruptured protein likely refolds back to its native state after the force is removed.

FIGS. 14A-14B show the AFM force curve of compressive force manipulation on a single protein molecule. FIG. 14A shows the AFM force curve of a single Apo-CaM molecule which shows a rupture event under compressive force. FIG. 14B shows the AFM force curve of a single Ca²⁺-ligated CaM molecule, which did not show any rupture event under compressive force manipulation.

Although the crystal structure of Ca²⁺-ligated calmodulin shows the central helix in α-helix form, the NMR structure conclusively shows this linker is nonhelical and very flexible around its middle point, from residue K77 to S81. The anisotropy observed for the motion of the two lobes was much smaller, which indicates that in Ca²⁺-ligated calmodulin, tumbling of both N terminal and C terminal lobes are mutually independent. This flexible central helix structure model is further supported by the structure of the CaM molecule complexed with target peptides, where these target peptides induce collapse of the elongated dumbbell structure forming a globular structure around the helical target peptide. In the presence of Ca²⁺ ions, this extended flexibility gives the CaM protein the conformational freedom where it can release the tension and avoid an abrupt rupture under compressive force. In other words, in the presence of Ca²⁺, a CaM molecule behaves more like a non-rigid sphere where it can easily change its shape redistributing the loading force applied by the AFM tip. As a result, the molecule does not go through a structural rupture under a compressive force.

On the other hand, in absence of Ca²⁺, the central helix of Apo-CaM is significantly less flexible which forbids both the domains to come together and bind to the peptide. The significantly larger degree of anisotropy in rotational diffusion observed for Apo-calmodulin relative to Ca²⁺-ligated calmodulin further shows that the linker is more rigid in the Apo state compared to the Ca²⁺ activated state. The Apo-CaM is in a more bounded state, which contributes to its structural rigidity whereas Ca²⁺-ligated calmodulin is in an open state. When the force reaches a threshold value of ˜70 pN, the CaM can no longer hold the force, and the molecule gets ruptured spontaneously and abruptly.

Protein rupture under compressive force is a spontaneous process driven by a threshold amount of force. As the AFM force loading is very slow at 1.5 nm/ms, temperature around the protein remains constant and the rupture dynamics follow a typical energy profile with an energy crossing barrier that includes the complex nature of dynamic bond breaking, intramolecular interaction dynamics, and liquid friction force. The inhomogeneous nature of the protein rupture indicates the inhomogeneous local environment constituted by different orientations of the protein molecules along with different electric, hydrophilic, and hydrophobic force fields of the single protein molecules, solvent molecules, and the linker molecules on the cover glass surface. Structural rigidity attributed from interdomain interactions, hydrogen bonds, and solvent dynamics is very important to study protein structure-function relationship associated with protein-protein, protein-peptide interactions, and enzymatic reactions.

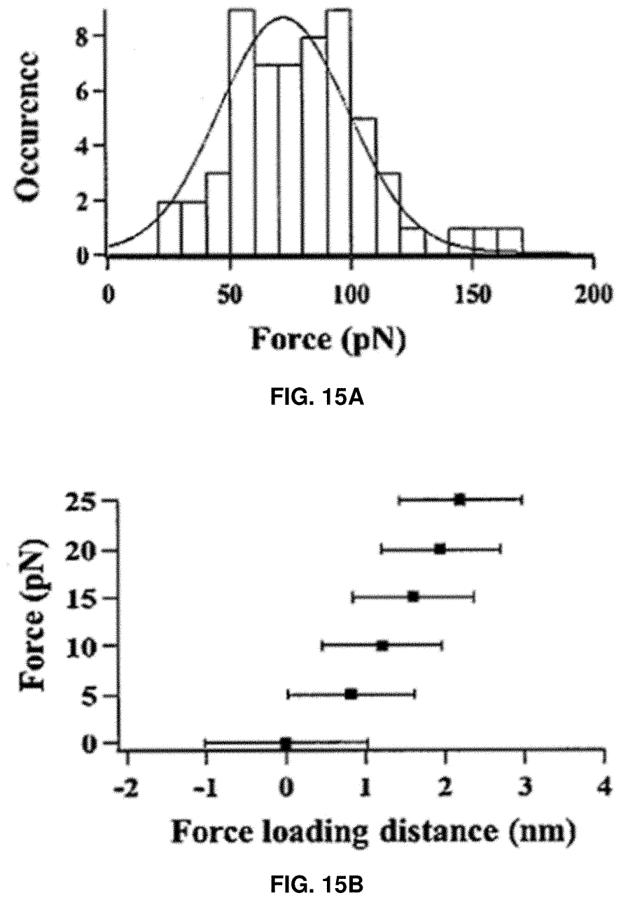

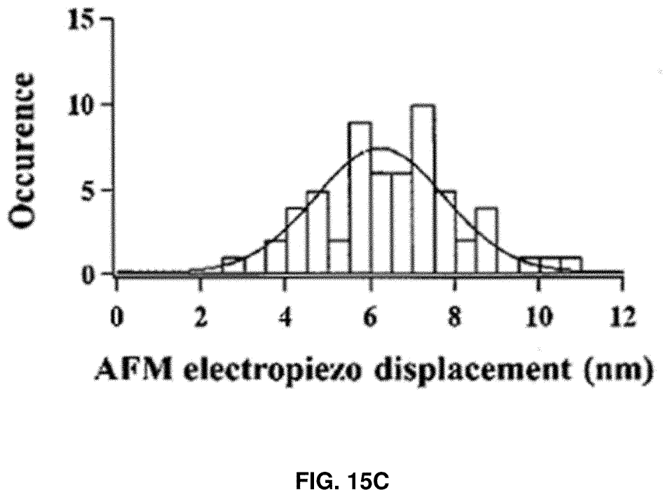

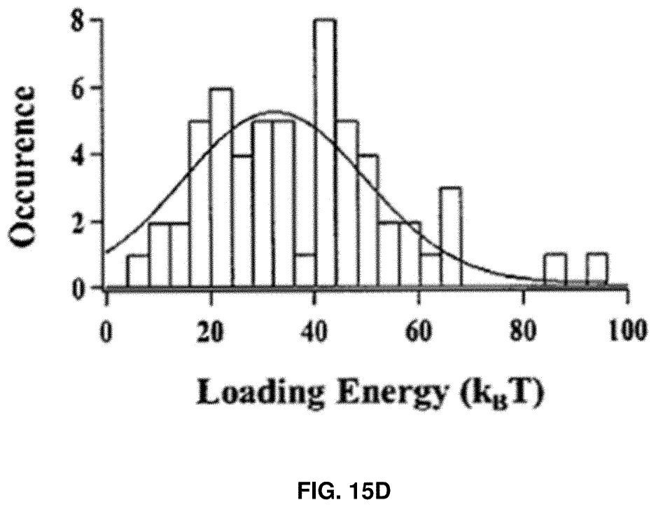

FIG. 15A shows the Gaussian distribution of the rupture force of single Apo-CaM molecules under 1500 nm/s approaching velocity. From this distribution, the rupture force was calculated to be around 70 pN. This rupture force is dependent on the AFM tip approaching velocity. To calculate the actual threshold force loading distance, i.e., the amount of structural change of the targeted Apo-CaM molecule under the process of compressive force loading to the threshold value, the force curves were analyzed considering two factors. First, the force loading process causes the tip bending. Because of that, the distance traveled by the electropiezo scanner exceeds the distance traveled by the AFM tip apex. The amount of tip bending in the process of force loading can be easily taken in account considering the force constant of the cantilever (30 pN/nm). Second, the contribution of the hydration shell around a protein molecule (1-2 nm of width) was considered. Therefore, in the recorded force curves, force increases in a much slower rate at the beginning which corresponds to the AFM tip interaction with the outer solvation layer of the protein. In the latter part of the force curve, the force increases in a much faster manner as the AFM tip starts to interact with the protein surface directly. Without wishing to be bound by theory, it is believed that the contact occurs when the compressive force loaded by the AFM tip reaches a certain value to counter the water solvation layer. Different assessments were plotted in FIG. 4B from a chosen “force upon contact” at 0, 5, 10, 15, 20, and 25 pN.

FIG. 15C shows the distribution of electropiezo displacement, which was read out directly from the force curve. It is the distance between the point where force loading starts and the point where protein rupture occurs. However, the actual distance of force loading to the threshold value is significantly shorter because of the electropiezo displacement calibration. The loading energy was calculated using E_{loading}=∫F(l)dl, where l is the compressive force loading distance, and F(l) is the loading force. Notably, the force loading distance is at least few nm less than the AFM electropiezo displacement (FIG. 15C) due to tip bending. Although the actual nature of the force loading curve on a single Apo-CaM is nonlinear, in the calculation it was assumed that force increases linearly during the loading process in the data analysis. Under this approximation, distribution of the calculated threshold compressive force loading energy was plotted in FIG. 15D using E_{loading}=(F threshold/2)×l_{threshold} and the mean value was around 32 k_{B}T; however, the energy can be as low as 10 k_{B}T with significant probability, which is biologically accessible from the local thermal fluctuations, such as in living mammalian cells at 37° C.

FIGS. 15A-15D show the characterization of compressive force loading. FIG. 15A shows the distribution of threshold force of Apo-CaM rupture under 1500 nm/s AFM tip approaching velocity. FIG. 15B shows the estimated average force loading distance after adjustment with standard deviation, under the assumption that the atomic level contact between AFM tip and the Apo-CaM molecule occurs after the force reaches 5, 10, 15, 20, and 25 pN, respectively, to counter the hydration shell (FIG. 15B). The negative sign represents the AFM tip position before contact with the protein solvation layer. The average threshold force loading distance was 3.8 nm. FIG. 15C shows the AFM electropiezo displacement distribution, which is defined as the distance traveled by AFM electropiezo from the start of the force loading on a targeted Apo-CaM molecule to the point of rupture. However, the actual distance of force loading is significantly shorter due to the electropiezo displacement calibration. FIG. 15D shows the distribution of calculated loading energy under 1500 nm/s loading velocity. All histograms (FIGS. 15A, 15C, 15D) are fitted with Gaussian function.

The structural flexibility and rigidity of the calmodulin molecule play a very important role in protein function associated with binding other proteins and peptides. Numerous attempts have been made over the years to address the flexibility of calmodulin in the presence and absence of Ca²⁺ using NMR and other techniques, but it is still hotly debated due to the absence of direct evidence. It is significant that the structural rigidity of a protein molecule can be probed by using a compressive force. It further proves that the compressive force is equally sensitive like a pulling force to sense such a miniscule amount of structural change in terms of flexibility and compactness. Furthermore, spontaneous single protein rupture of the bound Ca²⁺-deactivated form was also observed under compressive force, which may be a plausible mechanism leading to protein mis-folding and entangled aggregation.

The tau protein was also investigated. Tau is an intrinsically disordered protein (IDP) that plays a very important role in stabilizing axonal microtubules in the central nervous system. Tau 441 has two inserts at the N-terminal end, a proline-rich region, four repeats (R1, R2, R3, and R4), and a short C-terminal tail. In pathogenic form, tau loses its affinity towards the microtubule, and these four repeat domains fold together forming a β-sheet structure and aggregates as fibrillary tau. The role of tau protein in neurodegeneration is still unclear, but it is already known that tau mutation inducing tau aggregation is closely associated with tauopathies. It is believed that tau protein aggregation is transmittable in neurons in a prion-like manner.

Tau is a highly water-soluble protein and positively charged in physiological condition. NMR structure study indicates the structural diversity of tau protein in the solution and complicated network of transient long-range interactions. Due to the absence of a fixed tertiary structure, tau protein is believed to exist as an IDP in solution. Both circular dichroism measurements and electron paramagnetic resonance have shown a higher degree of mobility of the tau protein structure.

Both the molecular basis of early aggregation events and the mechanism by which tau aggregation causes neuronal dysfunction are still unclear. For aggregation, the IDP needs to undergo through conformational changes to form the “pro-aggregate”. It is observed that tau repeat domains K18 and K19 can aggregate much faster than the full-length tau. Tau mutation in frontotemporal dementia FTDP-17 is known for increasing β-sheet propensity making the structure more prone to aggregation.

Single molecule study has also shown evidence that tau protein is not completely lacking structural motifs. A Forster resonance, energy transfer study showed a much shorter intramolecular distance than a random coil model. A single molecular fluorescence polarization anisotropy study has shown that tau protein under solution exists in two conformations. These two long-lived conformations adopted by tau protein vary in terms of compactness.

In these examples, spontaneous tau protein rupture under compressive force manipulation using an atomic force microscope (AFM) is demonstrated. It was found that tau protein undergoes a spontaneous rupture when the compressive force reaches a threshold value depending on the tip approaching speed. It was also found that, in the presence of Mg²⁺ ions, tau protein does not show such rupture events, whereas the rupture event remains in the presence of monovalent cations like K⁺. This gives the indication of the presence of two forms of tau protein under different ionic environment. Under K⁺environment tau protein exists as a relatively rigid protein structure which is more prone to collapse under compressive force build up. With the addition of Mg²⁺ ions, the protein relaxes to a more flexible conformation that does not show any rupture. In addition, it was also investigated whether this kind of spontaneous and simultaneous rupture of multiple proteins under close proximity can develop an entangled protein third state, a fusion state, and can be a plausible mechanism for protein aggregation.

Compactness, rigidity, and force withholding are proportionally closely related concepts: they go high and low proportionally and monotonically. Compactness is more about the envelope of the protein outer sphere volume; rigidity is more about the protein inner structure collective stiffness and stability of the structure under thermal fluctuation; force withholding is about the force holding inside the protein structure, mostly reflected by stress, which is a direct consequence or association of the protein structure rigidity and compactness.

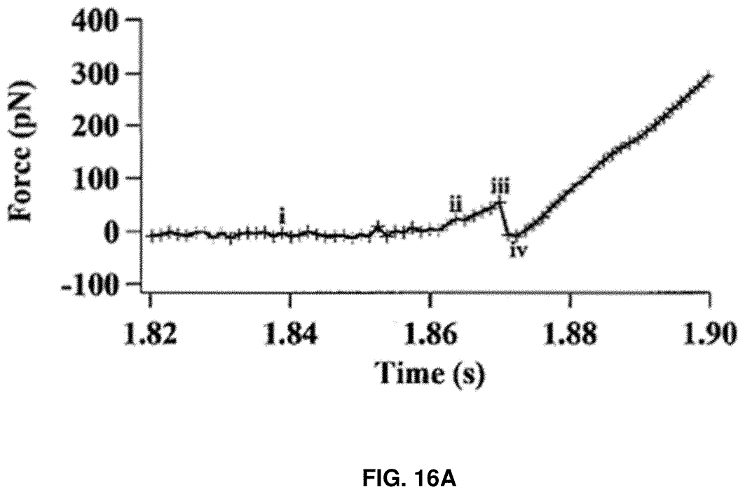

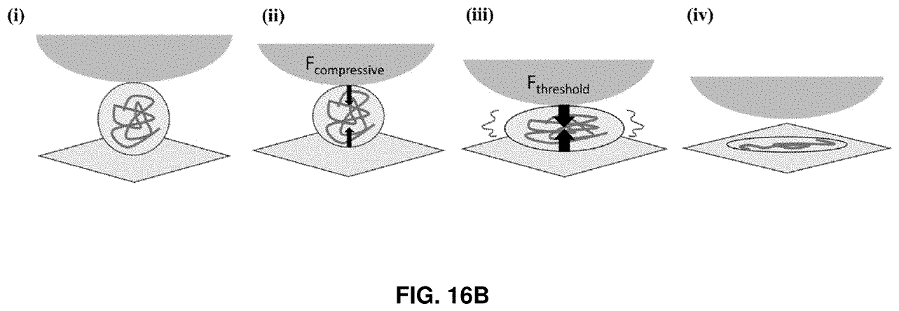

FIG. 16A shows the typical force curve of the AFM tip engagement with a single protein molecule and its response under an external compressive force. The protein molecules were tethered to the cover glass through covalent bonding between the amine group of the protein and the linker molecule attached to the coverglass surface. All the force curves were recorded under buffer solution (pH 7.4). When the AFM tip apex touches the surface of the protein molecule it starts loading force on it, once the force reaches a threshold value it can no longer withhold the force and collapses (FIG. 16A). This kind of behavior exists in globular proteins such as HPPK, calmodulin, and others.

This spontaneous protein rupture at the threshold compressive force reveals a unique hidden property associated with the rigidity of the tau protein structure in solution. Protein structure constituted with intermolecular hydrogen bonding, interdomain interactions, friction force with the solvent molecule and interdomain frictions can form structural rigidity which can withstand pN amount of compressive force applied by the AFM tip apex. When the compressive force reaches a threshold value the protein can no longer hold the force and gets ruptured. Though tau protein is intrinsically disordered protein in solution, it exists in different conformational states. These conformational states can be either long lived or short lived, and vary in terms of compactness. Ensemble average measurement gives the average picture of disordered protein structures in an averaged timescale, but the real picture of single molecular conformational fluctuation can be much more complex and different. Protein molecules can exist in a more rigid state for a time being, enabling the protein structure to withstand pN compressive force. Not only that, but increased hydrophobicity by the linker and the trimethoxy silane molecules can also induce the protein to form a globular structure. This increased molecular rigidity allows the AFM tip to load a pN scale of compressive force on the protein. Once the force reaches the threshold value, it causes multiple hydrogen bond rupture, and frictional force relaxation under intermolecular and protein-solvent interactions.

FIG. 16A shows a typical force curve of a tau protein rupture under compressive force. The sudden drop in the force curve represents the spontaneous structural rupture of the protein molecule. FIG. 16B shows a cartoon scheme of tau protein rupture under compressive force. (i) is the point where there is no physical contact between the AFM tip and the protein molecule. (ii) is where the molecule gets squeezed under the compressive force after the contact occurs. Black arrows represent the force vectors acting on the molecule, AFM tip, and the cover glass. (iii) is the point where the compressive force reaches the threshold value, the molecular interaction also becomes weakened to an amount that it no longer can hold the force, and as a result, the protein molecule gets ruptured. (iv) is the ruptured state of the protein.

This protein spontaneous rupture under compressive force is a complex event which includes inhomogeneous local factors like a hydrophilic hydrophobic force field of the protein molecules and the surrounding. This inhomogeneous nature of the protein rupture gives a broad distribution of the threshold force. The AFM force loading process is a relatively slow process compared to the protein conformational fluctuation time, which ensures that the rupture process follows an isothermal dynamic with a free-energy downhill kinetics. The scale of the rupture force at pN can be biologically available in living cells under the influence of thermal fluctuation, molecular crowding, and anisotropic force field. The anisotropic nature of force fluctuation inside the cells can generate pN compressive force that is capable of triggering such protein ruptures. A ruptured protein molecule can stay in a metastable or sub stable state and can refold back to its original folded state. Should more than one protein ruptured at the same time in a proximity, the ruptured proteins may fold into an entangled state, which may be an early event of protein aggregation. Nevertheless, it is highly remarkable that there may be a link between the protein rupture under compressive force and the protein aggregation.

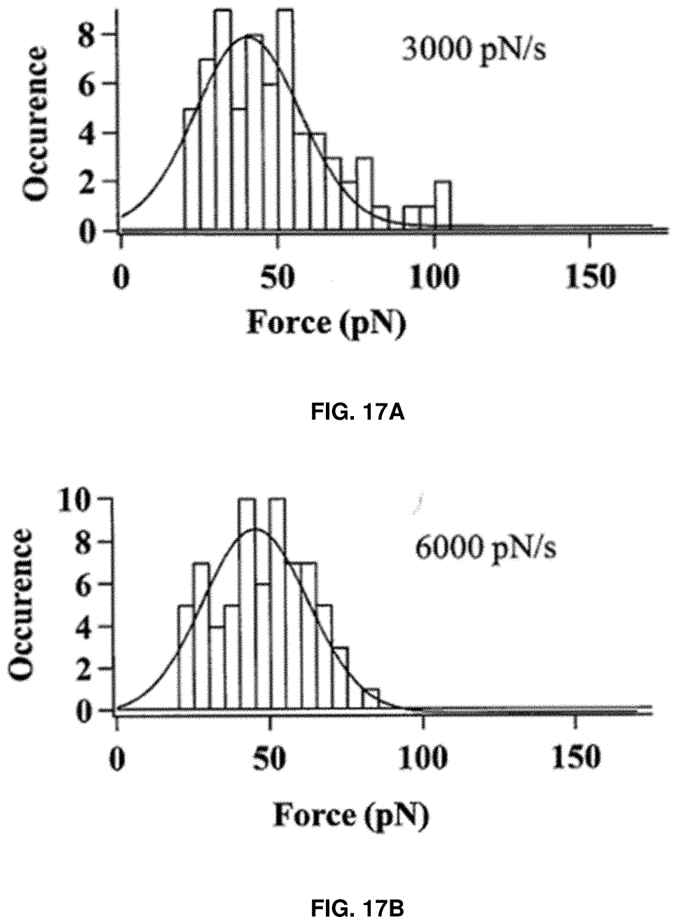

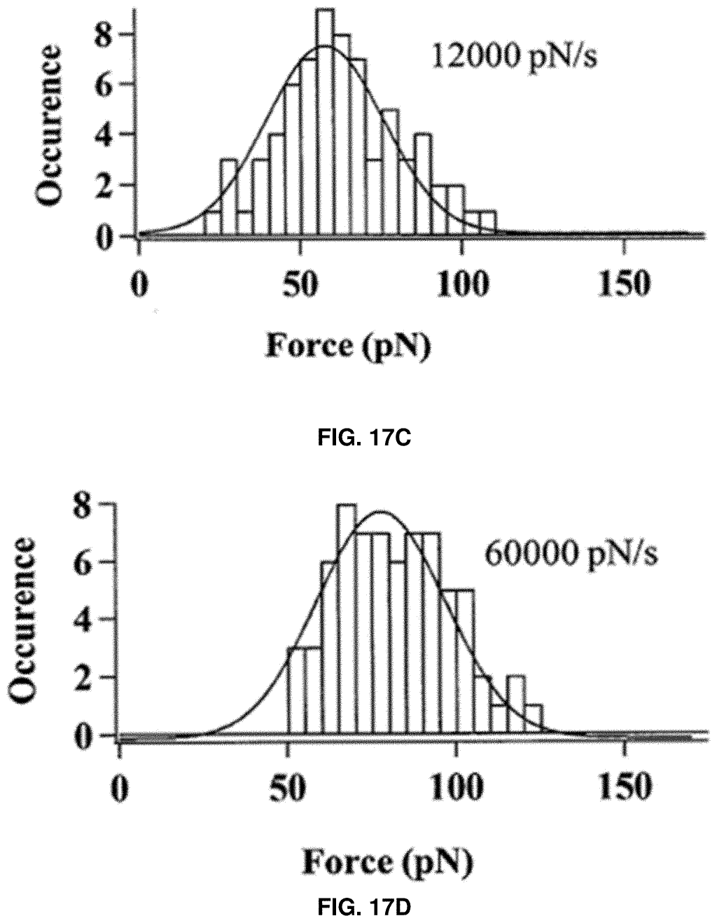

To further characterize the rupture threshold force distribution and mean rupture force, the experiment was repeated under the different approaching speed of the AFM tip. FIG. 17 shows the force loading speed dependence of the rupture force and the protein rupture threshold force distribution under different loading rates (3000, 6000, 12000, 60000 pN/s, respectively). The most probable rupture force was calculated after plotting histograms and fitting it with a Gaussian function. It was found that with the increase of the speed of the force loading the rupture force also increases from 40±2.33 pN to 78±2.04 pN.

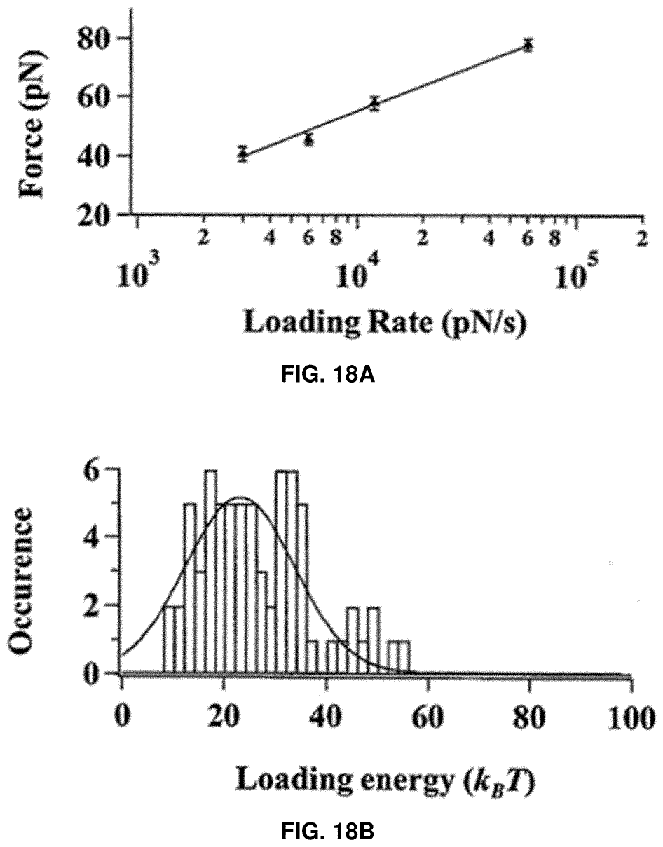

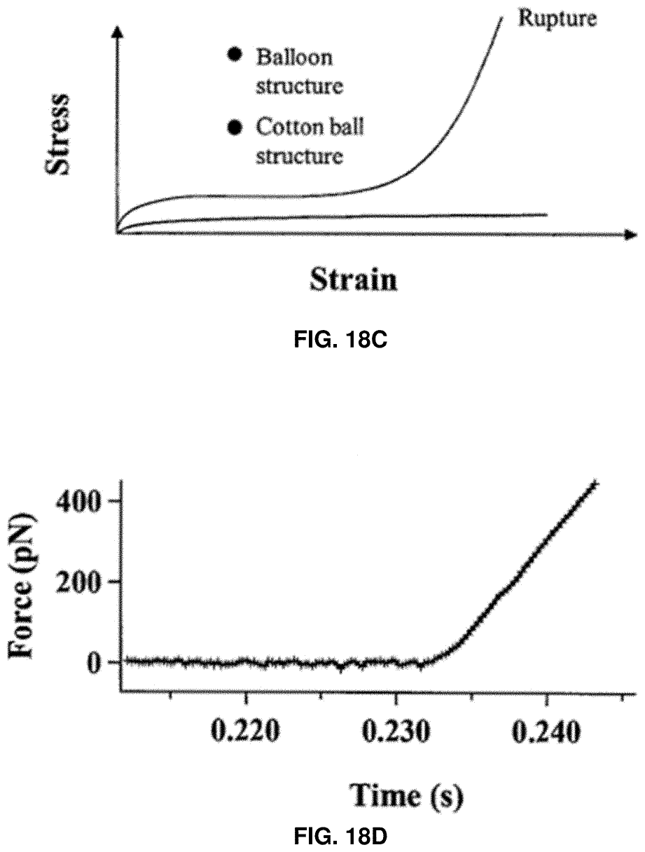

FIG. 18A shows the linear dependence of rupture force with the force loading rate. Notably, protein rupture under compressive force is a more complex process with a multi-dimensional force vector matrix. So, a linear fit may only hold in such a small range of loading rate. The actual picture may be very different including torsional force distribution inside the protein matrix induced by the AFM tip, multidimensional frictional force, and transient dipole interactions. Loading rate was calculated by multiplying the AFM tip speed with the force constant of the cantilever. FIG. 18B is the calculated loading energy under 3000 pN/s force loading rate. Where the E_{loading}=0.5*loading distance(1)*F_{threshold}. Though the loading energy is around 20 K_{B}T, few rupture events were around 10 K_{B}T, which indicates that this rupture events can also happen under thermal fluctuation.

To further investigate that whether the rupture events are closely related to the local environment, it was found that the protein rupture behaviors are highly sensitive to the charge of the cations in solution. As Mg²⁺ ions were added, no rupture event was found (FIG. 18D), which is different from the rupture behavior under K⁺ environment. Without wishing to be bound by theory, it is believed that there is a comprehensive model for two types of protein structure responses under compressive forces (FIG. 18C, green curve). One is a balloon-like type, reflected by the stress-strain curve in FIG. 18C, which is more rigid in nature and show rupture under compressive force. At the beginning of the force loading it starts changing both shape and build up the internal force, then only the strain changes without continued building up of internal force, then the strain changes slowed down but the stress built up increases up to the protein ruptures, like a balloon under a compressive force and eventually explodes. Whereas, the other is a cotton-ball-like type (FIG. 18C, red curve), which continues changing its shape under compression with no associated internal stress built up, therefore, ends up with no rupture under the compression. Increasing the ionic strength with the increased charge makes the hydrogen bond and the electrostatic attraction weaker. For example, Mg²⁺ ions relax the protein structure in a more flexible form than the K⁺ ions. Imbalance of certain metal ions found in neurodegenerative disorders indicate the possible involvement of metal ions in the process of neurodegeneration. The present examples show a possible relation between the electrochemical environment and protein rigidity, which can be closely related to protein aggregation and tauopathy.

FIG. 18A shows the linear fit of threshold force with loading rate. FIG. 18B shows the distribution of loading energy under 3000 pN/s force loading rate. FIG. 18C shows the conventional stress vs strain curve of two protein forms. FIG. 18D shows the typical compressive force curve on tau protein under Mg²⁺ environment.

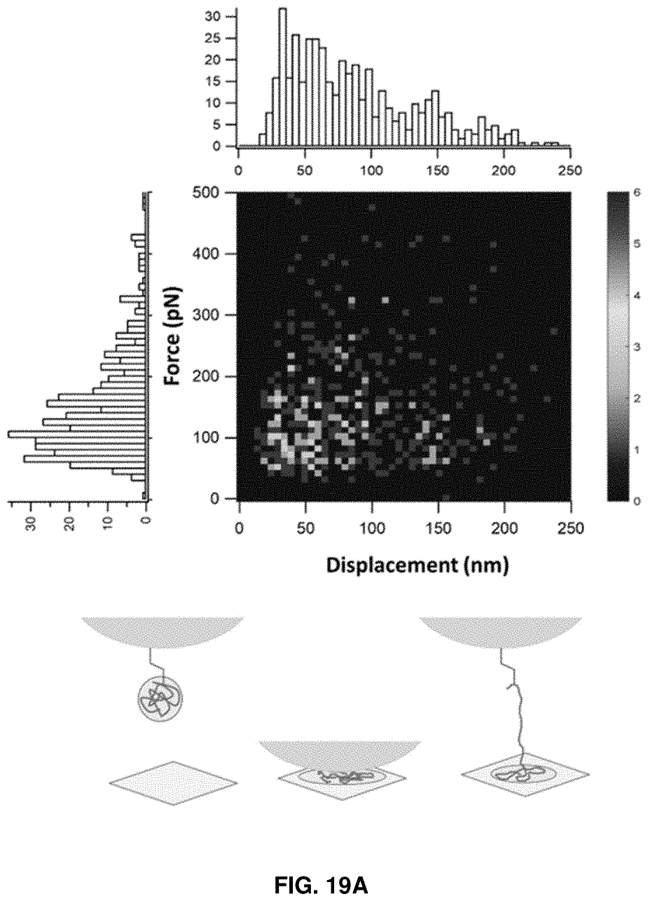

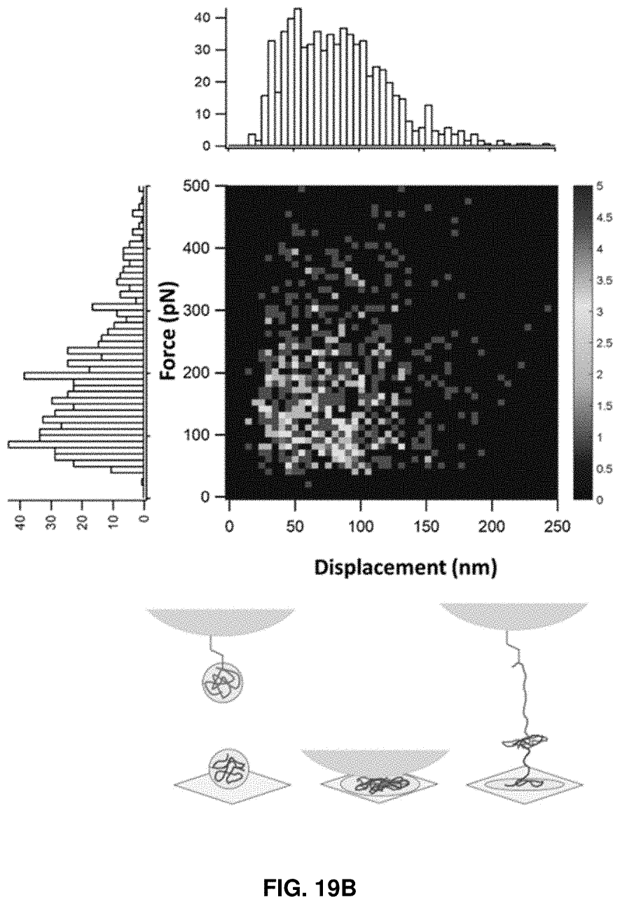

Protein rupture under compressive force can be irreversibly consequential inside the cell under protein crowding. A ruptured protein can stay in a metastable state, can refold back to its original state, or can stay in a different conformational state. Under molecular crowding, the compressive force fluctuation can trigger a rare event involving multiple protein ruptures, and the simultaneously ruptured protein cluster can fold up into an entangled aggregation. To demonstrate this mechanism, an AFM pulling experiment followed by compressive force loading was conducted, inducing a pair of protein ruptures. The AFM tip was covered with a monolayer of tau protein with a significantly short linker. A force matrix experiment was performed on a coverglass surface that was covered with tethered tau protein in a single molecular low concentration, where the AFM tip was approached by some points apart from each other by a certain distance, and both the approaching curve and the pulling curve was recorded. The experiment was performed under a buffer solution (pH 7.4) with 2000 nm/s approaching velocity. The pulling rupture force was measured and plotted against the displacement. FIG. 19A represents the 2D plot of force vs displacement pattern of control experiment where the tau protein coated AFM tip was approached by a trimethoxy silane coated coverglass with no tau protein molecules attached with it. And FIG. 19B is the force vs displacement pattern of the pulling rupture where a tau protein coated AFM tip was approached by a coverglass surface with attached tau protein on it. In the case of FIG. 19B a higher as well as broader pulling rupture force than the control experiment is seen. Not only that, but more frequent pulling rupture events in the case of FIG. 19B than in the case of FIG. 19A (almost double) are seen. The result indicates that there is a specific attraction or interaction present between a pair of ruptured tau proteins under the compressive force induction. These higher pulling force ruptures can also indicate the presence of an entangled form between the tau protein on the AFM and the tau protein on the cover glass. It is hard to distinguish one from the other because of the experimental limitations. But this is the first indication of the presence of tau-tau interaction under compressive force manipulation.

FIGS. 19A-19B show a 2D plot of force vs displacement of pulling rupture events. FIG. 19A shows control experiment with tau coated AFM tip and trimethoxy silane coated coverglass. Here the observed rupture force is due to the interaction between the tau protein with the surface, which is unspecific in nature. As a result, the force distribution was in the lower end and is also narrow in nature. Below is the cartoon scheme. FIG. 19B shows a distribution of pulling rupture events followed by a collapse of the tau coated AFM tip on a coverglass surface with attached tau protein molecules. Below is the cartoon scheme.

### Conclusion

There is a significant difference in the nature of the protein rupture dynamics under pulling force and compressive force. Protein rupture under pulling force is a force driven process in every step, whereas the protein rupture in the compressive experimental force application described herein is a spontaneous process initiated by a threshold force to put the protein matrix at the starting point of the spontaneous process. As the force loading is slow at 0.2 nm/ms, the compressive force loading is an isothermal process, and the protein remains at equilibrium with a constant temperature surrounding. The intrinsically isothermal dynamics follow the activation crossing barrier process under the liquid friction force and molecular intra-protein interaction network matrix. The protein rupture process under the threshold compressive force is a complex process involving binding and unbinding, dissociation and association, multiple coordinate force vectors, relaxation, and accumulation. Furthermore, it also involves complex factors, such as stochastic noncovalent interactions, conformational deformation, tension distribution within the targeted protein, surface adsorption, and desorption between the AFM tip and the protein molecule. Nevertheless, the real experimental local environment creates an energy landscape for the ruptured state of the protein, and the potential energy well is deeper than the metastable or far-from-equilibrium protein state under the threshold compressive force. The local environment is constituted by the protein's electric, hydrophilic, and hydrophobic force fields associated with different orientations of the protein on the cover glass surface, as well as the hydrocarbon modified surface of the coverglass. The local solvation layers are formed by the water and buffer electrolyte solution. Nevertheless, there is an inhomogeneous local environment that provides inhomogeneous protein rupture event energy landscapes, and in turn, gives inhomogeneous protein rupture energetics and dynamics.

It is remarkable that the structural rupture can be observed as a response of a globular protein under a compressive force directly applied upon it. This protein function and behavior are demonstrated and identified quantitatively in the correlated single-molecule AFM force manipulation and fluorescence imaging spectroscopy experiments described herein, and the observations cannot be achieved by conventional AFM imaging and force manipulation alone. Furthermore, the rupture threshold force is relatively weak, and the threshold compressive force loading energy is 4-30 k_{B}T, depending on different proteins. Moreover, the protein rupture behavior is also highly sensitive to the local environment as the rupture events are free energy driven and spontaneously occur when the compressive force reaches the threshold. The related biological impact on protein functions and protein dynamics can be highly extensive and profound, since the force fluctuations are capable of providing the threshold compressive force in living cells to trigger transient protein ruptures that may be followed by protein misfolding and entangled aggregations closely related to human neuronal degenerative diseases, such as brain chronic traumatic encephalopathy and neurofibrillary tangles.

Protein structure spontaneous rupture was observed under mechanical compressive force directly applied by the AFM-FRET single-molecule nanoscopic approach. The rupture of protein molecule under compressive force is an abrupt and spontaneous process, and the threshold force is rather weak, ranging from 12 pN to 75 pN, which is a biologically relevant force amplitude in living cells with significant biological consequences.

In addition, AFM compressive force was utilized to manipulate and characterize both calcium activated and deactivated forms of calmodulin. Upon loading of compressive force on a single Apo-CaM molecule, an abrupt and spontaneous rupture of the protein was observed, which is an unexplored property of the protein. On the other hand, no such events were observed in the case of the Ca²⁺-ligated form. The Ca²⁺-ligated form is more flexible, which makes it unable to hold force. This protein property is highly significant in protein functions in living cells, as the thermal fluctuation local force fluctuations may provide such a pN force and trigger a protein structure collapse or a multiple protein collapse simultaneously at the same location, a catastrophic unfolding event, that may be associated with protein dysfunction, aggregation, and misfolding.

Tau protein rupture was observed under pN of compressive force, which indicates the presence of some rigid conformational states of an intrinsically disordered protein. This rigidity may be the possible reason for tau aggregation. It was also found that structural rigidity of a protein is closely related to the electrochemical environment. In the presence of Mg²⁺, tau protein exists in a more relaxed and flexible form than in a K⁺ environment. This indicates an imbalance of certain ions can impact the rigidity of a protein, which can trigger the protein aggregation.

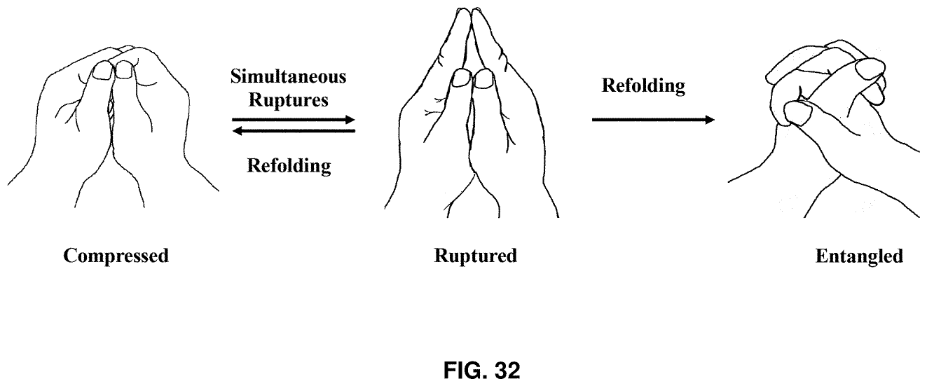

It is very important to have a molecular understanding of the earliest steps of the protein aggregation. Here an understanding of the protein aggregation process under influence of compressive force in a crowded environment is described. Simultaneously ruptured proteins in crowding can either refold back to their individual native states or refold into an entangled state as both pathways are energetically accessible. (FIG. 32.) The AFM pulling force experiment indicates the presence of specific interactions between tau proteins or intermolecular entanglement under compressive force.

The examples herein show that by changing the concentration of cation, one can prevent or reduce the amount or frequency of protein rupture when that protein is subjected to a compressive force. The experimental conditions described above indicate that proteins in living cells are also subjected to compressive forces and, likewise, that specific cation concentrations can prevent or reduce ruptures. Accordingly, the present disclosure is useful in evaluating new drug candidates or as a treatment itself.

Ions have a huge variety of roles in cells. Ions that can be used in accordance with the present disclosure include ions involved in electrical communication (Na⁺, K⁺, Ca²⁺), as cofactors in dictating protein function with entire classes of metalloproteins (constituting, by some estimates, at least ¼ of all proteins), in processes ranging from photosynthesis to human respiration (Mn²⁺, Mg²⁺, Fe²⁺), as a stimulus for signaling and muscle action (Ca²⁺), and as the basis for setting up transmembrane potentials that are then used to power key processes such as ATP synthesis (H⁺, Na⁺). In addition, organic ions can be used in some embodiments.

Concentrations of ions used in accordance with the present disclosure can be within physiological ranges. Physiological ranges usually means, but is not limited to, concentrations in the millimolar range. One skilled in the art will understand that this range will depend, for example, on the ion itself, the type of cell or fluid in which the ion is present, and the physiological state of that cell (e.g., dormant or excited neuros will have different concentrations of certain cations). These examples are one of many factors known to those skilled in the art involved in determining what is a physiological concentration of ion. Many outside references address this complicated topic, such as, but not limited to, “Cell Physiology Source Book: Essentials of Membrane Biophysics,” 4th Edition, ed. Nicholas Speralakis, 2012, Academic Press, which is hereby incorporated by reference for all purposes.

### METHODS

### Chemically Linking HPPK Molecules on a Cover Glass

The cover glass (Gold Seal) was first cleaned and silanized with a mixture of (3-aminopropyl) trimethoxysilane and isobutyltrimethoxysilane with a ratio of 1:10000 dissolved in DMSO (10% v/v) for 12 hours, and then incubated in 10 nM dimethyl suberimidate in 50 mM PBS (pH 8.0) for 4 h. After washing by water and methanol, the glass slide was incubated in 10 nM HPPK solutions (PBS, pH 7.4) for 4 h. The possibility of two protein molecules locating underneath the AFM tip or in the same laser focus spot of around 300 nm diameters is almost zero. Cy3-Cy5 labeled HPPK molecules were tethered randomly on a cover glass surface and then incubated in PBS buffer (pH=7.4) in a homemade chamber immobilized on top of the microscope. To decrease photobleaching, added 0.8% D-glucose, 1 mg/mL glucose oxidase, 0.04 mg/mL catalase, and about 1 mM Trolox were added in the buffer as an oxygen scavenger. To make sure that only one specific single-molecule HPPK molecule was probed at a time, the HPPK molecules were tethered to the glass surface with diluted concentration by controlling the ratio of the mixture of (3-aminopropyl) trimethoxysilane and isobutyltrimethoxysilane, and 1:10000 is the ratio that was used to ensure that the possibility of two or more HPPK molecules within an area defined by the diffraction limit is virtually zero.

### Single Molecule Measurement or Manipulation

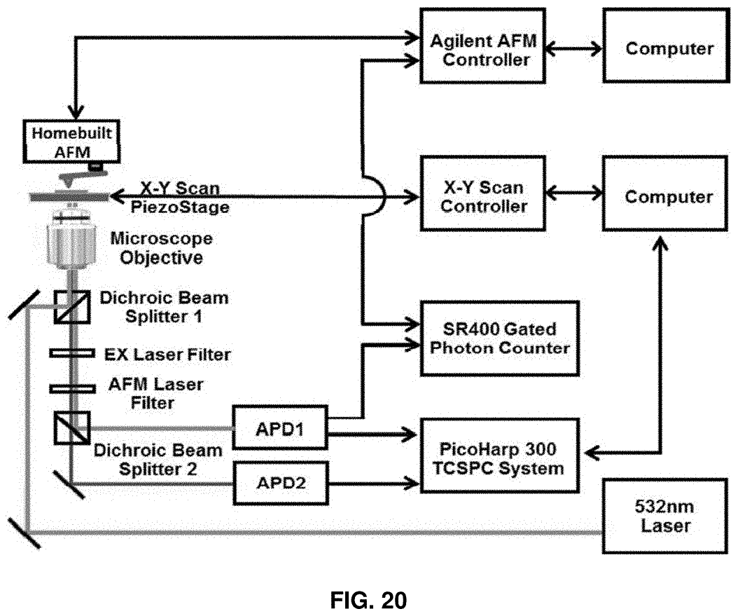

After making the AFM tip coaxially aligned with the excitation laser, the x-y position of the AFM tip was fixed, and moving the sample stage alone can allow for switching different single molecules and implementing further measurements. Due to instrumental drifting, further routine adjustments were undertaken, but they were relatively minor compared to the initial alignment. The whole process for the correlated AFM-FRET alignment is shown in FIGS. 20-24 (described below).

### AFM-FRET Correlated Nanoscopy Setup

The home-built experimental setup was composed primarily of an inverted optical microscope (Axiovert-200, Zeiss) and an AFM scanning module (PicoSPM, Agilent) in an over-under configuration. The excitation laser (532 nm) beam was reflected by a dichroic beam splitter (z532rdc, Chroma Technology) and focused by a high-numerical-aperture objective (1.3 NA, 100X, Zeiss) on the sample surface at a diffraction limited spot of about 300 nm in diameter. To obtain a single-molecule FRET image and photon-counting time trajectories, the emission signal was split using a dichroic beam splitter (640dcxr) into two-color beams centered at 570 nm and 670 nm representing the emissions of the Cy3 and Cy5 donor-acceptor dye pair, respectively. The two-channel signals were collected by a pair of Si avalanche photodiode single photon counting modules (SPCM-AQR-16, Perkin Elmer Optoelectronics) for detecting the single-molecule fluorescence. It was possible to obtain a fluorescence image (ranging from 1 μm×1 μm to 100 μm×100 μm, typically 10 μm×10 μm) by continuously raster-scanning the sample over the laser focus with a piezoelectric scanning stage (Physik Instruments Inc., Germany) at any scanning speed (typically ranging from 1 ms/pixel to 30 ms/pixel), with each image being normally 100 pixels×100 pixels. Typically, fluorescence intensities of the FRET donor (Cy3) and acceptor (Cy5) were collected for several hundred seconds by a two-channel Picoharp 300 (PicoQuant) time-correlated single photon counting (TCSPC) system.

A manual two-axis x-y mechanical positioning stage (Zeiss) and a two-axis close loop x-y 100 μm piezoelectric-scanner stage (Physik Instruments) were mounted directly on the optical microscope. The two-axis close loop x-y piezoelectric-scanner stage was controlled by a computer with a raster scan software, by which the sample was able to be scanned over laser in two-dimension (2D) to provide images and identify positions of dye-labeled single-molecule proteins within the laser focal spot. The two-axis x-y mechanical positioning stage was used to support the close-loop AFM scanning module (PicoSPM, Agilent). With that, the AFM tip was moved in 2D on top of the x-y piezoelectric-scanning stage independently, and the AFM tip was positioned to co-axial with the laser beam from the microscope objective. An AFM scanner was used to scan topographic images or manipulate single molecules by force. A home-built fluid cell was put on top of the sample (a transparent glass cover-slide) to keep the sample in buffer solution. To avoid the FRET signal from being interfered by AFM laser, the AFM scanning module was modified by an infrared superluminescent diode (SLD) at 950 nm to replace the conventional 650 nm laser source. A shortpass filter E835sp (OMEGA Optical) was put in front of the detectors to block the AFM infrared photons and a longpass filter HQ5451p (Chroma Technology) was put to block 532 excitation laser: M: Mirror, Dichroic beam splitter 1: z532rdc (Chroma Technology), reflecting 532 nm excitation laser beam and transmitting fluorescence. Dichroic beam splitter 2: 640dcxr (Chroma Technology), splitting the emission signal into two color beams centered at 570 nm and 670 nm representing Cy3 and Cy5 emissions. APD 1: Si avalanche photodiode single photon counting modules (SPCM-AQR-16, Perkin Elmer Optoelectronics) for detecting the single-molecule fluorescence Cy5. APD 2: Si avalanche photodiode single photon counting modules (SPCM-AQR-16, Perkin Elmer Optoelectronics) for detecting the single-molecule fluorescence Cy3. Filter 1: HQ5451p (Chroma Technology), blocking 532 nm excitation laser beam. Filter 2: E835sp (OMEGA Optical), blocking AFM infrared 950 nm beam.

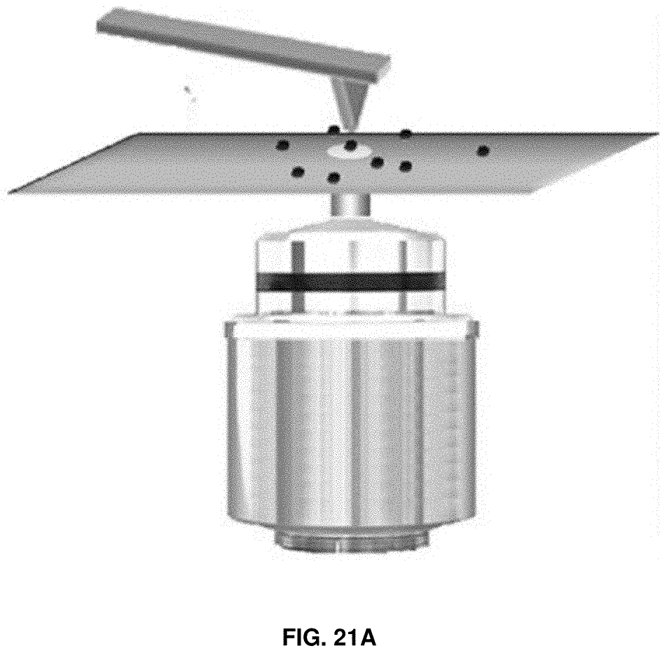

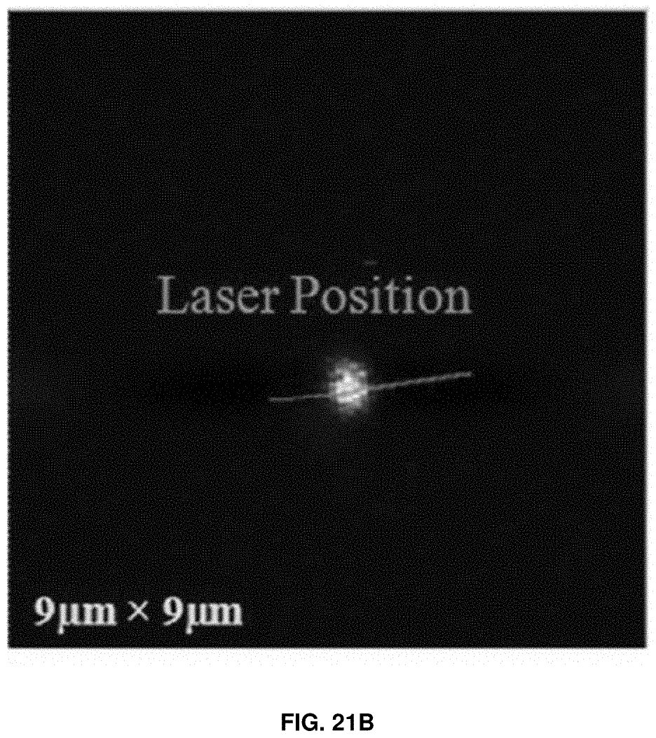

FIGS. 20-21 show the experimental setup of single-molecule AFM-FRET nanoscopy. For the AFM topological imaging and single-molecule force manipulation, a typical contact AFM tip coated with Cr/Au was utilized to obtain force spectroscopy of the protein underneath. To collect data from one single molecule at a time from both spectroscopic and AFM amplitude channels simultaneously, the optical focal point and AFM tip were spacially lined up precisely so that AFM tip, laser beam focus, and target molecule were in the co-axial alignment.

Lining up the optical focal point and AFM tip is the first and critical step for a typical operation of the AFM-FRET nanoscopy. First, the x-y two-axis mechanical positioning stage was moved to roughly align the AFM tip with the laser beam focal point by observing the reflection pattern of the AFM tip; a symmetric light reflection pattern can be observed from the microscope objective. It indicates that the coaxial position is achieved within a few micrometers.

To co-axially align the AFM tip with the laser beam center of Gaussian distribution of the laser focus, the AFM tip is scanned across the area of the laser beam that has been aligned, and one of the APD signals is sent to the AFM controller through a gated photon counter SR400 (Stanford Instruments, CA) as shown in FIG. 21A. The image of the optical intensity was taken during AFM tip scanning. FIG. 21B shows a bright spot due to the photons from tip reflection as the AFM tip scans over the laser beam, because the tip can be considered as a micro mirror that reflects more photons back through the objective, effectively enhancing the photon collection solid angle of the microscope. Through this alignment, the AFM tip can be aligned with the center of the laser beam to within a hundred nanometers.

FIG. 20 shows the schematic diagram of coaxial laser and AFM tip. FIG. 21A shows the AFM tip scans right on the protein and the laser beam focus spot. FIG. 21B shows an optical image of laser focus spot under AFM tip scanning. The bright spot indicates the laser beam position.

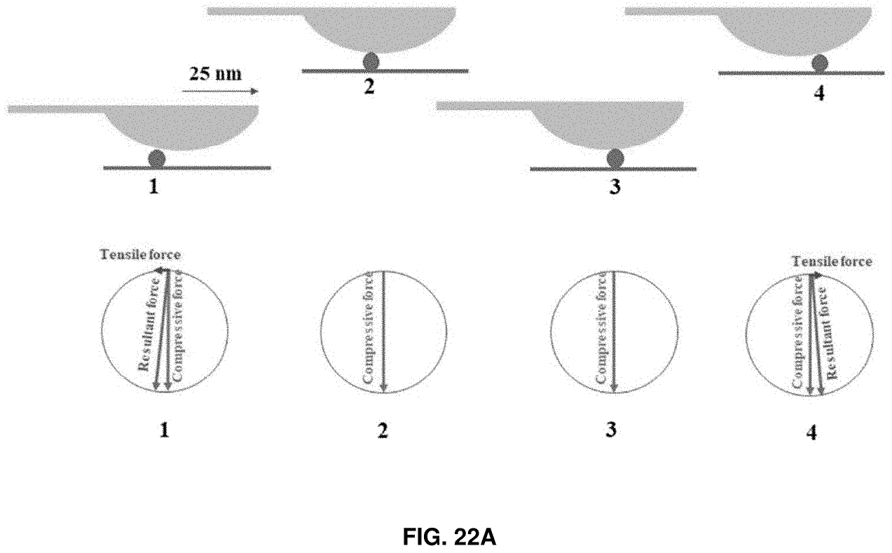

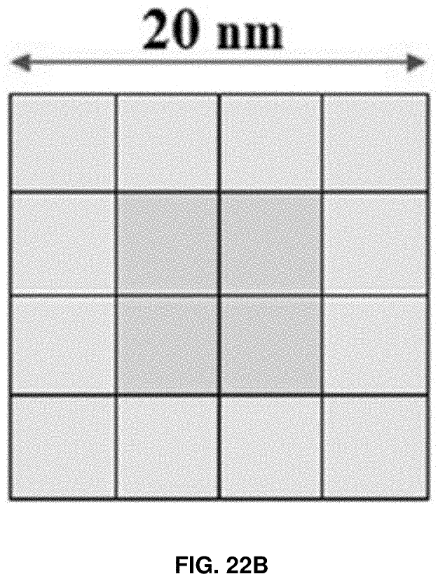

FIGS. 22A-22B show single molecule measurement and force mapping. After the AFM tip alignment with excitation laser, the reflection is strongest when the apex of the tip is right on top of the molecule. Therefore, by moving the AFM tip to the center of the image, AFM tip, the target single molecule, and the laser focus point are aligned as precisely as tens of nanometers. After these procedures, the x-y position of the AFM tip is to be fixed, and moving the sample stage alone can allow for switching different single molecules and implementing further measurements. After the alignment, a sample stage scanning is conducted to look for single protein molecules on the modified glass surface. The quality of the alignment can also be justified by the detected photon counting level of the resulting image. The optical signal from dye labeled single HPPK molecule would be much stronger with the AFM tip right on top of the laser focal point. Even though the AFM tip, laser focus, and the molecule are coaxially aligned, the molecule can be away from the AFM tip by tens of nanometers as the size of the protein molecule is much smaller than the diffraction limit of the fluorescence light. So, to ensure direct contact of the AFM tip apex surface with the molecule, a 16×16 matrix covering 300×300 nm² area was made under the laser focus where the AFM force curve measurement was taken in every 20 nm interval to ensure a protein is under the AFM tip for a correlated force-manipulation and FRET imaging analysis. As the typical radius of the coated AFM-tip apex is less than 35 nm, and the size of the HPPK molecule is only about 5 nm in diameter, a 20×20 nm² area can be divided in total sixteen 5×5 nm² pixels where a protein molecule can be found in any of these pixels. As depicted in FIGS. 22A-22B, protein molecules can be oriented under an AFM tip in four possible ways. Out of those, in two of the cases protein molecule can be compressed without any traces of tensile force, whereas in the cases of ‘1’ and ‘4’ there can be very little to a negligible amount of tensile force acting on the protein molecule. In this case, it is most likely that the compressive force reaches the protein rupture threshold force value, but the tensile force is still too small to make any impact on the protein except some possible deformation, which may be the reason that a broad distribution of the threshold compressive force for the protein ruptures is seen. Nevertheless, force manipulation on a protein molecule represents either one of these four possibilities, making it a compressive force measurement.

FIGS. 22A-22B show the force mapping on a single protein molecule. FIG. 22A shows four possible positions of a protein molecule under the AFM tip and the different assessment of the acting force on the protein molecule. FIG. 22B represents the total number of ways a protein molecule can be located under the AFM tip. The green box represents where the protein molecule can be compressed without any trace of tensile force, whereas the blue box represents where the protein molecule can be compressed with a very small to negligible amount of tensile force.

While the AFM tip is repeatedly pushed down and pulled up, multiple force spectroscopic data and FRET trajectories are collected simultaneously, and they are recorded in the same temporal axis. The FRET efficiency was calculated by the donor acceptor intensity using the formula EFRET=IA/(IA+ID), where IA is the fluorescence intensity of the acceptor and ID is the fluorescence intensity of the donor. As the AFM tip was moving down and closer to the laser focal point, the detected photon counts in both donor and acceptor channels rose due to a previously reported micro-mirror reflection effect, resulting in a higher signal collection efficiency. Typically, the micro-mirror effect appears or disappears at 150 nm above the sample surface.

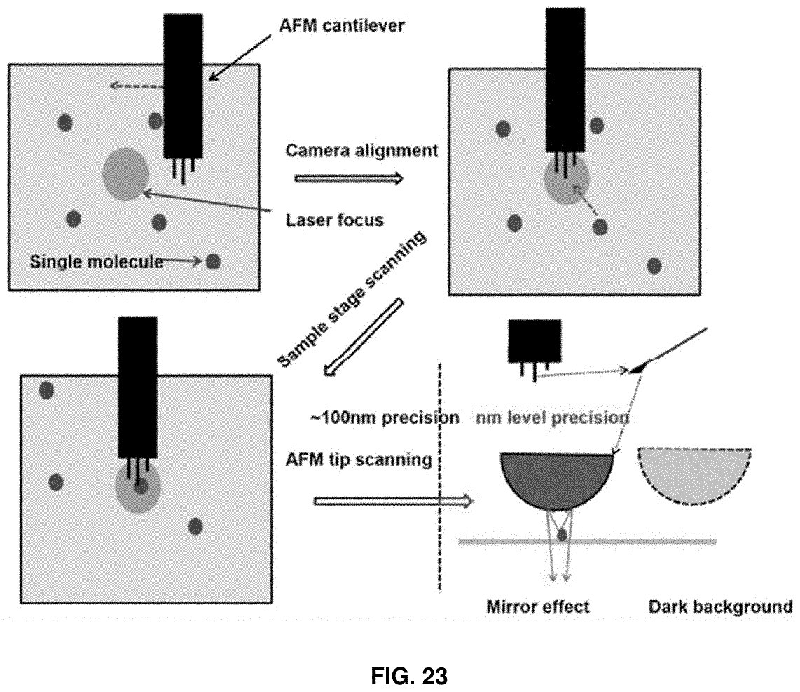

FIG. 23 shows the procedures of AFM-focus-point alignment in the AFM-FRET correlated measurement. The black rectangle represents the AFM cantilever. The green circle represents the laser focus point. The size of the laser focus in the figures is drawn larger than actual size relatively. The red spots represent single protein molecules immobilized on the cover glass.

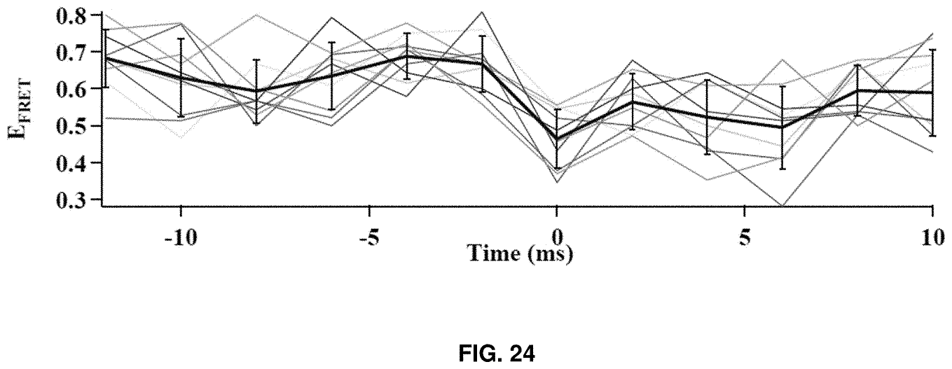

FRET intensity trajectories correlate with compressive force curve measurements. FIG. 24 shows different colors representing the ten different FRET intensity trajectories correlated with compressive force curve measurement. (−)ve time represents the time before rupture, (+)ve time represents the time after rupture, and time zero represents the moment of rupture. The black line is the average of all the trajectories with standard deviation error bar.

The Pearson's product-moment correlation coefficient analysis was used between FRET efficiency E(t) and measured force F(t) (eqn. 1):

$${r}_{F , E} ( T , n ) = \frac{1}{n - 1} {\sum }_{t = T - ( n - 1 ) \cdot 2 ms}^{t = T} ( \frac{F ( t ) - \overline{F} ( t )}{{s}_{F} ( T )} ) ( \frac{E ( t ) - \overline{E} ( T )}{{s}_{E} ( T )} ) \qquad ( 1 )$$

where T is the index of FRET efficiency time trajectory based on 2 ms bin; n is the number of data points within each calculation window (12 data points were used in the calculation (FIG. 6E), starting from 10 ms time bin before the rupture event and continues to 10 ms time bins after the rupture event, where rupture event occurs at t=0; (t) is force measured; E(t) is FRET efficiency calculated based on the smFRET measurements; F̅(T), Ē(T) are the sample averages within each calculation window; s_{F}(T) and s_{E}(T) are the sample standard deviations within each calculation window, which is defined as in (eqn. 2).

$${s}_{x ( T , n )} = \sqrt{\frac{1}{n - 1} {\sum }_{t = T - ( n - 1 ) \cdot 2 ms}^{t = T} {( x ( t ) - \overline{x} ( T ) )}^{2}} \qquad ( 2 )$$

FIG. 6E shows that the Pearson's product-moment correlation coefficient reaches the maximum (close to 1) at the moment of the rupture events which implies higher correlation. The distribution of the coefficients appears to be narrowly distributed at the time of rupture, indicating a stronger temporal correlation between the force drop and the protein rupture; whereas, the distribution is much broader away from the rupture event time (FIG. 6E). Although each rupture event can have different force trajectory in terms of rupture threshold force value and force loading time, the release of compressive force and sudden decrease of FRET efficiency are highly correlated statistically.

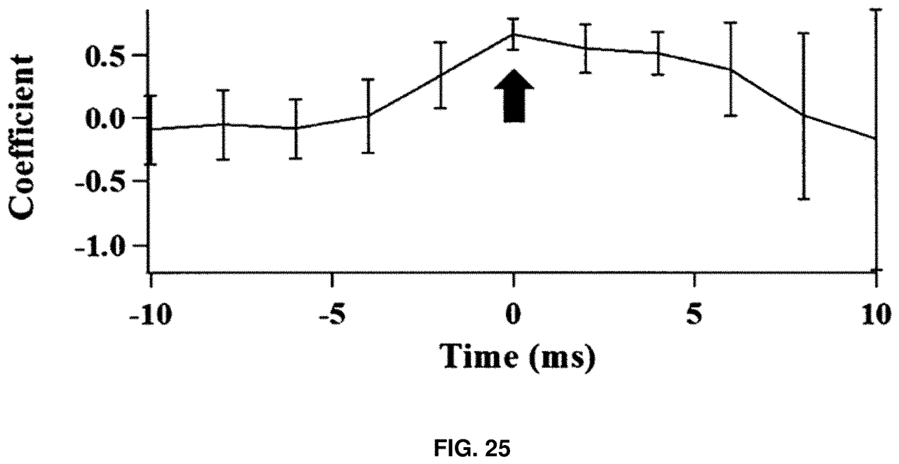

FIG. 25 shows the mean of all the thirteen trajectories of FIG. 6E, with standard deviation error bar. The blue arrow indicates the point of rupture where the standard deviation error bar is narrow, and the value is close to 1, indicating a high correlation of force drop and the FRET efficiency drop at the protein rupture event.

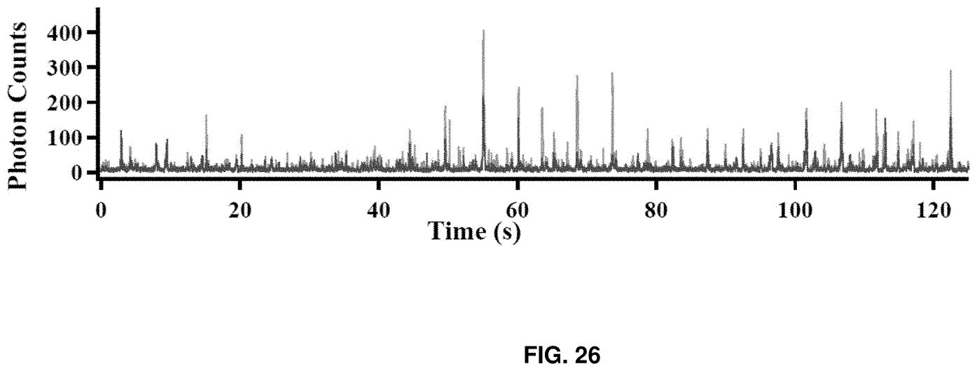

A control experiment for the signal-to-noise ratio of the fluorescence photon detection by the correlated single-molecule AFM-FRET nanoscope was performed. After the AFM tip was positioned coaxially with the excitation laser focus, the sample was raster scanned in 10 ms/pixel, and the fluorescence intensity data were collected during the scanning (FIG. 26). The signal-to-noise ratio of the fluorescence measurement was calculated by comparing the peak signals (as the fluorescence molecule crossover the scanning laser focal point) and the baseline signal (no fluorescence molecule under the scanning laser focal point).

FIG. 26 shows one scanning period of the fluorescence intensity-time trajectory for the signal-to-noise ratio control experiment of the AFM-FRET nanoscopy setup, showing the FRET donor (green) and acceptor (red) channel signals. The baseline is about 10 counts/10 ms, and the peaks signal are mainly distributed in the range from 100 to 150 counts/10 ms. So, the signal-to-noise ratio for the setup is in the range from 10 to 15.

The loading energy was calculated using E_{loading}=J F(l)dl. l is the compressive force loading distance, and F(l) is the loading force curve. The force constant of AFM cantilever=30 pN/nm, and the average rupture threshold force is 47 pN under 200 nm/s force loading velocity. In the calculation, the force being linearly increased during loading process was considered, which gives F(l) =F_{threshold}. Under the above approximation, the distribution of calculated threshold compressive force loading energy is plotted for HPPK and CaM, given k_{B}T=4.114 pN.nm. Most of the rupture events are triggered by loading energy around 30 kBT for HPPK and 4-8 k_{B}T for CaM protein.

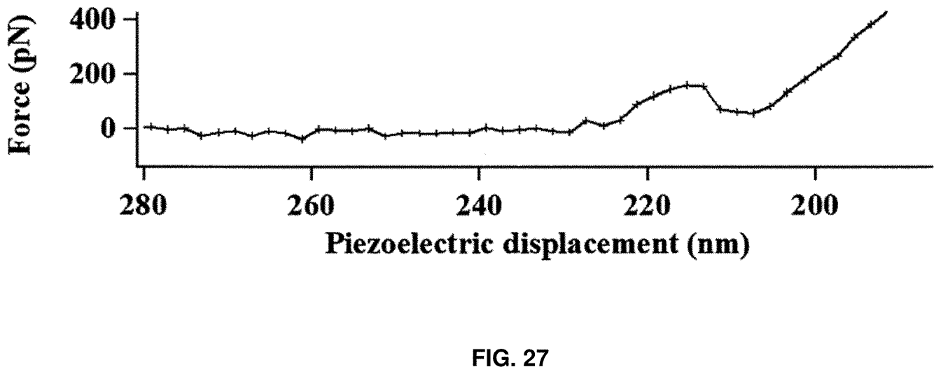

FIG. 27 shows the same force trajectory of FIG. 6A (single HPPK protein rupture) plotted with AFM piezoelectric displacement.

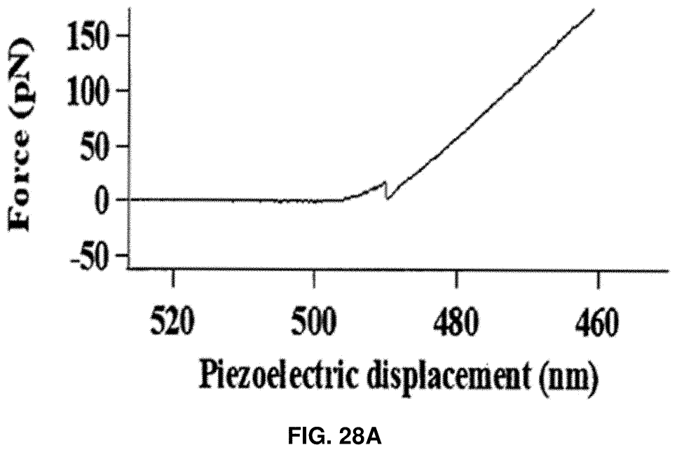

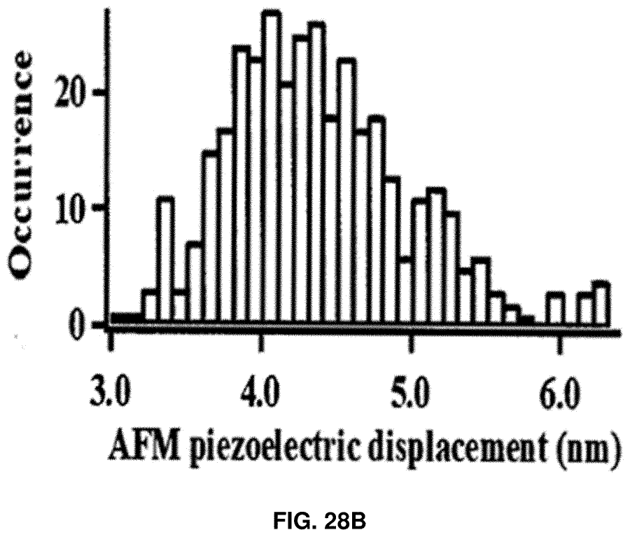

FIG. 28A shows force vs AFM piezoelectric displacement data of a single CaM protein rupture. FIG. 28B shows a distribution of AFM piezoelectric displacement under 100 nm/s loading velocity, which is defined as the distance traveled by AFM electropiezo between the start of the force loading on a targeted protein and the protein rupture.

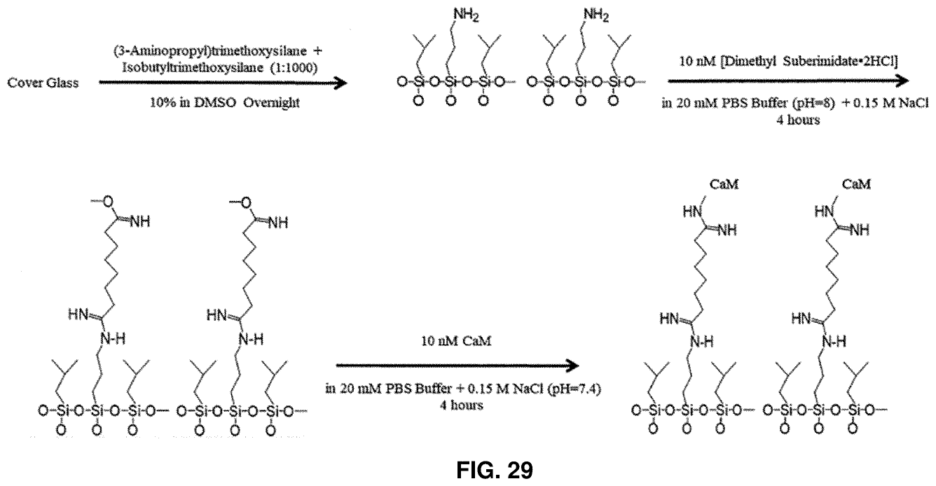

FIG. 29 illustrates the scheme of tethering single protein molecules on a cover glass surface. The cover glass (Gold Seal) was first cleaned and silanized with a mixture of (3-aminopropyl) trimethoxysilane, isobutyltrimethoxysilane with a ratio of 1:1000 dissolved in DMSO (10% v/v) for 12 hours, and then incubated in 10 nM dimethyl suberimidate in 50 mM PBS (pH 8.0) for 4 h. After washing with water and methanol, the glass slide was incubated in 10 nM CaM solutions (PBS, pH 7.4) for 4 h. Apo-CaM molecules were tethered randomly on a cover glass through amidine bond with amino groups of the protein and then incubated in HEPES buffer (pH=7.4) for the AFM force curve experiment. 1 mM concentration of CaCl₂ was added to generate saturated Ca²⁺ environment allowing the single-molecule Apo-CaM convert to Ca²⁺0 ligated CaM. To make sure that only one specific single-molecule CaM molecule was probed at a time, the CaM molecules were tethered to the glass surface with a diluted concentration by controlling the ratio of the mixture of (3-aminopropyl) trimethoxysilane, isobutyltrimethoxysilane.

### CaM Conformational Changes

Immobilized CaM was incubated in an EGTA-containing buffer (20 mM HEPES, 2 mM EGTA, pH 8) at room temperature for 15 minutes and then treated with PBS buffer (pH 7.4) for five minutes. This process was repeated for four times to remove the Ca²⁺ ion from CaM, inducing CaM to change to the Apo-CaM conformation.

Typical compressive force curves were recorded under different conditions. FIG. 30 shows a compressive force curve on a calmodulin molecule under different Ca²⁺ conditions. The top graph is a typical force curve of a compressive force loading process on an Apo-CaM molecule. Force drop represents the protein rupture event. The middle graph is a typical force curve of AFM tip approach to a targeted calmodulin molecule under Ca²⁺ environment. No force drop was recorded, which represents the absence of rupture events. The bottom graph is a typical force curve of a compressive force loading process on a targeted calmodulin molecule after EGTA treatment. As EGTA removes Ca²⁺ ions from CaM molecule changing it to Apo-CaM, the rupture event recovers and was recorded in the force curve.

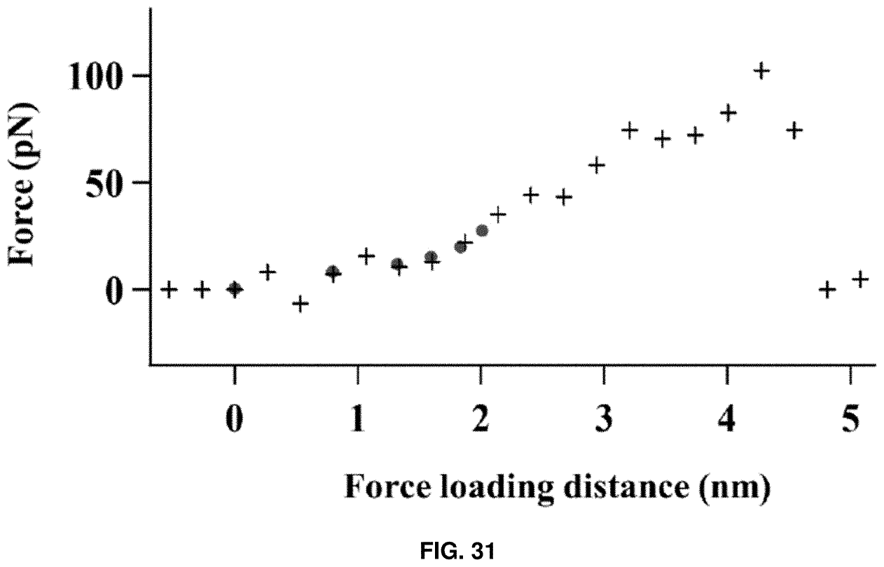

A force vs force loading distance curve is shown in FIG. 31, depicting an example of experimental force curve on a single Apo-CaM molecule. The small red dots show the points where force just exceeds 0 pN, and reaches 5, 10, 15, 20, and 25 pN, respectively. ‘0’ nm represents the point where the compressive force starts loading, i.e., the contact between the AFM tip and the protein solvation layer.

In accordance with the provisions of the patent statutes, the present invention has been described in what is considered to represent its preferred embodiments. However, it should be noted that the invention can be practiced otherwise than as specifically illustrated and described without departing from its spirit or scope.

Certain embodiments of the compositions and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

What is claimed is:

1. A method for preventing or reducing rupture of a protein from a compressive force, the method comprising exposing the protein to a concentration of a cation effective to prevent or reduce rupture of the protein from the compressive force.

2. The method of claim 1, wherein the protein is present in a human cell.

3. The method of claim 1, wherein the protein is a globular protein.

4. The method of claim 1, wherein the protein is selected from the group consisting of tau, HPPK, nitric oxide synthase (NOS), and calmodulin (CaM).

5. The method of claim 1, wherein the cation is selected from the group consisting of metallic cations and organic cations.

6. The method of claim 1, wherein the cation comprises Ca²⁺.

7. The method of claim 1, wherein the cation comprises Mg²⁺.

8. The method of claim 1, wherein the cation comprises Zn²⁺.

9. The method of claim 1, wherein the compressive force is at least about 12 pN.

10. The method of claim 1, wherein the protein is in an aqueous solution.

11. The method of claim 10, wherein the aqueous solution is present in a living cell of a subject selected from the group consisting of human, mammal, and other animal.

12. The method of claim 11, wherein the subject has a neurodegenerative disease or other condition involving protein misfolding, dysfunction, or aggregation.

13. The method of claim 1, wherein the concentration of the cation is a micromolar concentration.

14. The method of claim 1, wherein the concentration of the cation is a millimolar concentration.

15. A method of causing a spontaneous protein rupture, the method comprising applying a picoNewton compressive force to a protein to cause the protein to spontaneously rupture.

16. The method of claim 15, wherein the picoNewton compressive force is applied by a tip of an atomic force microscope.

17. The method of claim 15, wherein the applied compressive force is at least 20 pN.

18. The method of claim 15, wherein the applied compressive force is at least 25 pN.

19. The method of claim 15, wherein the applied compressive force is at least 60 pN.

20. The method of claim 15, wherein the applied compressive force is at least 75 pN.

21. An aqueous solution comprising:
a protein with a rupture threshold exposed to a compressive force in excess of the rupture threshold; and
a concentration of a cation;
wherein the concentration of the cation is sufficient to prevent rupture of the protein upon exposure to the compressive force in excess of the rupture threshold, and the protein is in a non-ruptured state.
